(19) United States
(12) Patent Application Publication
(10) Pub. No.: US 2016/0220574 A1
(43) Pub. Date: Aug. 4, 2016

(54) NSAID AND SIGMA RECEPTOR LIGAND COMBINATIONS

(71) Applicant: LABORATORIOS DEL DR. ESTEVE, S.A., Barcelona (ES)

(72) Inventors: Daniel ZAMANILLO-CASTANEDO, Barcelona (ES); Enrique PORTILLO-SALIDO, BARCELONA (ES); Enrique-José COBOS-DEL-MORAL, GRANADA (ES)

(21) Appl. No.: 15/021,050

(22) PCT Filed: Sep. 11, 2014
(86) PCT No.: PCT/EP2014/069370

§ 371 Date: Mar. 10, 2016

(30) Foreign Application Priority Data
Sep. 12, 2013 (EP) 13382353.4

(51) Int. Cl. A61K 31/5377; A61K 31/192; A61K 31/167; A61K 31/415; A61K 31/196; A61K 31/635

(52) U.S. Cl. CPC A61K 31/5377; A61K 31/196; A61K 31/635; A61K 31/167; A61K 31/415; A61K 31/192

(57) ABSTRACT

The invention refers to a synergistic combination comprising a Sigma ligand, particularly a Sigma ligand of general formula (I), and NSAID compound, a medicament comprising said active substance combination, and the use of said active substance combination for the manufacture of a medicament, particularly for the prophylaxis and/or treatment of pain.

### FIELD OF THE INVENTION

The present invention relates to an active substance combination, particularly for the prophylaxis and/or treatment of pain.

### BACKGROUND

The treatment of pain conditions is of great importance in medicine. There is currently a world-wide need for additional pain therapy. The pressing requirement for a specific treatment of pain conditions is documented in the large number of scientific works that have appeared recently in the field of applied analgesics.

PAIN is defined by the International Association for the Study of Pain (IASP) as “an unpleasant sensory and emotional experience associated with actual or potential tissue damage, or described in terms of such damage” (IASP, Classification of chronic pain, 2nd Edition, IASP Press (2002), 210). Although it is a complex process influenced by both physiological and psychological factors and is always subjective, its causes or syndromes can be classified. Pain can be classified based on temporal, aetiological or physiological criteria. When pain is classified by time, it can be acute or chronic. Aetiological classifications of pain are malignant or non-malignant. A third classification is physiological, which includes nociceptive pain (results from detection by specialized transducers in tissues attached to A-delta and C-fibres), that can be divided into somatic and visceral types of pain, and neuropathic pain (results from irritation or damage to the nervous system), that can be divided into peripheral and central neuropathic pain. Pain is a normal physiological reaction of the somatosensory system to noxious stimulation which alerts the individual to actual or potential tissue damage. It serves a protective function of informing us of injury or disease, and usually remits when healing is complete or the condition is cured. However, pain may result from a pathological state characterized by one or more of the following: pain in the absence of a noxious stimulus (spontaneous pain), increased duration of response to brief stimulation (ongoing pain or hyperpathia), reduced pain threshold (allodynia), increased responsiveness to suprathreshold stimulation (hyperalgesia), spread of pain and hyperalgesia to uninjured tissue (referred pain and secondary hyperalgesia), and abnormal sensations (e.g., dysesthesia, paresthesia).

Nonsteroidal antiinflammatory drugs (NSAIDs) are used to assist in the management of various chronic pain syndromes (Herndon et al., 2008). As a group, these medications are the most widely used medications in the world (Dugowson et al., 2006). Pain relief and decreased inflammation produced by NSAIDs result from suppression of the COX function of prostaglandin H synthase and the consequent formation of prostaglandin E2 (PGE(2)) and prostaglandin 12 (prostacyclin). Both cyclooxygenase-1 and -2 are expressed in the spinal cord, and the spinal COX product PGE(2) contributes to the generation of central sensitization upon peripheral inflammation. Further, spinal COX inhibition is also considered an important mechanism of antihyperalgesic pain treatment (Telleria-Diaz et al., 2010).

Clinical indications of NSAIDs include a variety of rheumatologic conditions, such as ankylosing spondylitis and rheumatoid arthritis. Osteoarthritis involves at least intermittent inflammation and can also respond to NSAIDs. Most importantly, local inflammation routinely occurs in response to acute injury of virtually any structure in the body. Thus, NSAIDs are a logical choice for acute pain management after injury. NSAIDs are widely used in the treatment of acute musculoskeletal injuries, and there is evidence for their ability to provide symptomatic relief of conditions such as acute low back pain. NSAIDs are also commonly used in chronic musculoskeletal pain, although the rationale for their use in that setting is less clear because the degree to which inflammation plays a role in chronic musculoskeletal pain is not known. The literature on the efficacy of NSAIDs in chronic musculoskeletal pain is mixed (Dugowson et al., 2006; Herndon et al., 2008). However, therapeutic utility of NSAIDs is limited by undesirable adverse effects including cardiovascular and gastrointestinal toxicity, for example producing ulcers (Dugowson et al., 2006; Herndon et al., 2008).

Acetaminophen, also known as paracetamol, can also be considered as a nonsteroidal anti-inflammatory drug with potent antipyretic and analgesic actions but with very weak anti-inflammatory activity. Debate exists about its primary site of action, which may be inhibition of prostaglandin (PG) synthesis (COX-1, COX-2 or putative COX-3) or through an active metabolite influencing cannabinoid receptors (Botting-RM, 2000).

Also the mechanism of action of metamizole (dipyrone) is not entirely clear. Unlike the acidic nonsteroidal anti-inflammatory drugs (NSAIDs), metamizole produces analgesic effects associated with a less potent anti-inflammatory action in different animal models. Therefore it has been proposed that the antinociceptive effect of dipyrone is mediated at least in part by central mechanisms (Hinz et al., 2007).

Two subtypes of Sigma receptors (Sigma-1 and Sigma-2 receptors) have been identified (Cobos et al., 2008). Confused with opioid receptors for many years due to the cross-reactivity of some ligands, the Sigma-1 receptor is a 24-kDa molecular mass protein of 223 amino acids anchored to the endoplasmic reticulum and plasma membranes (Cobos et al., 2008; Maurice and Su, 2009). Sigma-1 receptor is a unique ligand-regulated molecular chaperone which is activated under stress or pathological conditions and interacts with several neurotransmitter receptors and ion channels to modulate their function. The effects reported preclinically with Sigma-1 receptor ligands are consistent with a role for Sigma-1 receptor in central sensitization and pain hypersensitivity and suggest a potential therapeutic use of Sigma-1 receptor antagonists for the management of neuropathic pain as monotherapy (Romero et al., 2012).

Pyrazole derivatives of general formula (I) according to the present invention are described in WO 2006/021462 as compounds having pharmacological activity towards the sigma (σ) receptor useful, inter alia, in the prophylaxis and/or treatment of pain.

Pharmaceutical compositions (WO 2011/064296 A1), salts (WO 2011/064315 A1), polymorphs and solvates (WO 2011/095579 A1), and other solid forms (WO 2012/019984 A1) of said sigma ligands of formula (I) have been also disclosed as well as combinations with other active substances such a with opioids or opiates (WO 2009/130310 A1, WO 2012/016980 A2, WO 2012/072782 A1) or with chemotherapeutic drugs (WO 2011/018487 A1, WO 2011/144721 A1).

As mentioned above, therapeutic utility of NSAIDs is limited by undesirable adverse effects including cardiovascular and gastrointestinal toxicity (Dugowson et al., 2006; Herndon et al., 2008). Thus, strategies aimed to reduce doses needed for NSAIDs analgesia are desirable, in order to improve their therapeutic window and extend their use in clinics.

### BRIEF DESCRIPTION OF THE INVENTION

It is an object of the present invention to provide a medicament suitable for the prophylaxis and/or treatment of pain, which preferably does not show the undesired side effects of the NSAIDs when used for the prophylaxis and/or treatment of pain, or at least less frequent and/or less pronounced.

The inventors of the present invention have found and demonstrated that the administration of some specific Sigma receptor ligands in conjunction with NSAIDs surprisingly potentiates synergistically the analgesia.

In particular, the inventors of the present invention have found and demonstrated that the administration of some specific Sigma receptor ligands in conjunction with NSAIDs potentiates synergistically the analgesic effect of the latter, indicating that the combination of a Sigma ligand and a NSAID reduces the doses of the latter needed to obtain effective analgesia.

Furthermore, the inventors of the present invention have found and demonstrated that the administration of some specific Sigma receptor ligands in conjunction with NSAIDs potentiate synergistically the analgesic effect of Sigma ligands.

In particular, the Sigma ligands according to the present invention are Sigma-1 receptor ligands.

More particularly, the Sigma ligands according to the present invention are selective Sigma-1 antagonist receptor ligands. Preferably, the Sigma ligands according to the present invention are selective Sigma-1 antagonist receptor ligands of below defined formula (I) or a pharmaceutically acceptable salt, isomer, prodrug or solvate thereof.

Therefore, one aspect of the present invention relates to a synergistic combination comprising at least one NSAID and at least one Sigma ligand.

In a preferred embodiment, the at least one sigma ligand has a general formula (I), or a pharmaceutically acceptable salt, isomer, prodrug or solvate thereof

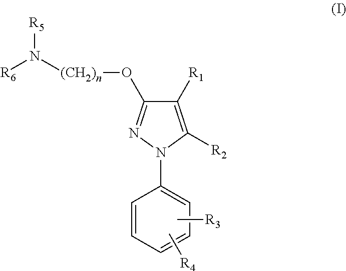

wherein,
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR_{B}, —OC(O)R₈, —S(O)ₜ—R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR_{E}, —C(O)OR₈, —C(O)NR₈R₉, —CH═NR₈, —CN, —OR_{B}, —OC(O)R₈, —S(O)ₜ—R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR_{E}, —C(O)OR₈, —C(O)NR₈R₉, —CH═NR₈, —CN, —OR_{B}, —OC(O)R₈, —S(O)ₜ—R₈, —NR₈R₉, —NR₈C(O)R₉, —NO₂, —N═CR₈R₉, and halogen, or together with the phenyl they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₈, —CH═NR₈, —CN, —OR_{B}, —OC(O)R₈, —S(O)ₜ—R₈, —NR₈R₈, —NR₈C(O)R₈, —NO₂, —N═CR₈R₈, and halogen;
or together form, with the nitrogen atom to which they are attached, a substituted or unsubstituted, aromatic or non-aromatic heterocyclyl group;
n is selected from 1, 2, 3, 4, 5, 6, 7 and 8;
t is 0, 1 or 2;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen.

A further aspect of the invention refers to a Sigma ligand as defined above for use in synergistically potentiating the analgesic effect of a NSAID when said NSAID is used in the prophylaxis and/or treatment of pain.

Another aspect of this invention refers to the use of a Sigma ligand as defined above for manufacturing a medicament for synergistically potentiating the analgesic effect of a NSAID when said NSAID is used in the prophylaxis and/or treatment of pain.

Another aspect of this invention refers to the combination comprising at least one Sigma ligand as defined above and at least one NSAID for use in the prophylaxis and/or treatment of pain.

Another aspect of this invention refers to the use of the combination comprising at least one Sigma ligand as defined above and at least one NSAID for manufacturing a medicament for the prophylaxis and/or treatment of pain.

Another aspect of the invention is a method of treatment and/or prophylaxis of a patient suffering from pain, or likely to suffer pain, the method comprising administering to the patient in need of such a treatment or prophylaxis a therapeutically effective amount of a combination comprising at least one Sigma ligand as defined above and at least one NSAID.

The pharmaceutical combination of the invention may be formulated for its simultaneous, separate or sequential administration.

In a preferred embodiment of the present invention, pain refers specifically to “post-operative pain”.

These aspects and preferred embodiments thereof are additionally also defined hereinafter in the detailed description, as well as in the claims.

### BRIEF DESCRIPTION OF THE FIGURES

FIG. 1: Potentiation of Diclofenac analgesia (0.625 mg/kg) by BD1063 (10, 20, 40 and 80 mg/kg) in the mechanical allodynia of the post-operative pain model in rats. n=10, *: p<0.05; ns: p>0.05 Dunnett, BD1063+Diclofenac vs. Diclofenac.

FIG. 2: Potentiation of Celecoxib analgesia (0.625 mg/kg) by BD1063 (10, 20, 40 and 80 mg/kg) in the mechanical allodynia of the post-operative pain model in rats. n=10, *: p<0.05; ns: p>0.05 Dunnett, BD1063+Celecoxib vs. Celecoxib.

FIG. 3: Potentiation of Paracetamol analgesia (20 mg/kg) by BD1063 (10, 20, 40 and 80 mg/kg) in the mechanical allodynia of the post-operative pain model in rats. n=10, *: p<0.05; ns: p>0.05 Dunnett, BD1063+Paracetamol vs. Paracetamol.

FIG. 4: Potentiation of Metamizole analgesia (0.156 mg/kg) by BD1063 (10, 20, 40 and 80 mg/kg) in the mechanical allodynia of the post-operative pain model in rats. n=10, *: p<0.05; ns: p>0.05 Dunnett, BD1063+Metamizole vs. Metamizole

FIG. 5: Potentiation of Diclofenac analgesia (0.625 mg/kg) by compound 63 (10, 20, 40 and 80 mg/kg) in the mechanical allodynia of the post-operative pain model in rats. n=10, *: p<0.05; ns: p>0.05 Dunnett, compound 63+Diclofenac vs. Diclofenac.

FIG. 6: Potentiation of Paracetamol analgesia (20 mg/kg) by compound 63 (5, 10, 20, 40 and 80 mg/kg) in the mechanical allodynia of the post-operative pain model in rats. n=10, *: p<0.05; ns: p>0.05 Dunnett, compound 63+Paracetamol vs. Paracetamol.

FIG. 7: Potentiation of Metamizole analgesia (0.156 mg/kg) by compound 63 (5, 10, 20, 40 and 80 mg/kg) in the mechanical allodynia of the post-operative pain model in rats. n=10, *: p<0.05; ns: p>0.05 Dunnett, compound 63+Metamizole vs. Metamizole.

FIG. 8: Potentiation of Celecoxib analgesia (0.625 mg/kg) by compound 63 (10, 20, 40 and 80 mg/kg) in the mechanical allodynia of the post-operative pain model in rats. n=10, *: p<0.05; ns: p>0.05 Dunnett, compound 63+Celecoxib vs. Celecoxib.

FIG. 9: Potentiation of Ibuprofen analgesia (0.625 mg/kg) by compound 63 (10, 20, 40 and 80 mg/kg) in the mechanical allodynia of the post-operative pain model in rats. n=10, *: p<0.05; ns: p>0.05 Dunnett, compound 63+Ibuprofen vs. Ibuprofen.

FIG. 10: Potentiation of Naproxen analgesia (0.312 mg/kg) by compound 63 (5, 10, 20 and 40 mg/kg) in the mechanical allodynia of the post-operative pain model in rats. n=10, *: p<0.05; ns: p>0.05 Dunnett, compound 63+Naproxen vs. Naproxen.

### DETAILED DESCRIPTION OF THE INVENTION

The efficacy of active components can sometimes be improved by addition of other (active) ingredients. More rarely, the observed efficacy of a combination of ingredients can be significantly higher than what would be expected from the amounts of the individual ingredients used, thus indicating potentiation of the activity of the components of the combination.

The present inventors have found that Sigma receptor ligands are able to synergistically potentiate the analgesic effect of NSAIDs.

In the context of the present invention, the following terms have the meaning detailed below.

“Alkyl” refers to a straight or branched hydrocarbon chain radical containing no unsaturation, and which is attached to the rest of the molecule by a single bond. Typical alkyl groups have from 1 to about 12, 1 to about 8, or 1 to about 6 carbon atoms, e. g., methyl, ethyl, n-propyl, i-propyl, n-butyl, t-butyl, n-pentyl, etc. Alkyl radicals may be optionally substituted by one or more substituents such as aryl, halo, hydroxy, alkoxy, carboxy, cyano, carbonyl, acyl, alkoxycarbonyl, heterocyclyl, amino, nitro, mercapto, alkylthio, etc. If substituted by aryl, it corresponds to an “arylalkyl” radical, such as benzyl or phenethyl. If substituted by heterocyclyl, it corresponds to a “heterocyclylalkyl” radical.

“Alkenyl” refers to a straight or branched hydrocarbon chain radical containing at least two carbon atoms and at least one unsaturation, and which is attached to the rest of the molecule by a single bond. Typical alkenyl radicals have from 2 to about 12, 2 to about 8 or 2 to about 6 carbon atoms. In a particular embodiment, the alkenyl group is vinyl, 1-methyl-ethenyl, 1-propenyl, 2-propenyl, or butenyl.

“Alkynyl” refers to a straight or branched hydrocarbon chain radical containing at least two carbon atoms and at least one carbon-carbon triple bond, and which is attached to the rest of the molecule by a single bond. Typical alkynyl radicals have from 2 to about 12, 2 to about 8 or 2 to about 6 carbon atoms. In a particular embodiment, the alkynyl group is ethynyl, propynyl (e.g. 1-propynyl, 2-propynyl), or butynyl (e.g. 1-butynyl, 2-butynyl, 3-butynyl).

“Cycloalkyl” refers to an alicyclic hydrocarbon. Typical cycloalkyl radicals contain from 1 to 3 separated and/or fused rings and from 3 to about 18 carbon atoms, preferably from 3 to 10 carbon atoms, such as cyclopropyl, cyclohexyl or adamantyl. In a particular embodiment, the cycloalkyl radical contains from 3 to about 6 carbon atoms.

“Aryl” refers to single and multiple ring radicals, including multiple ring radicals that contain separate and/or fused aryl groups. Typical aryl groups contain from 1 to 3 separated or fused rings and from 6 to about 18 carbon ring atoms, such as phenyl, naphthyl (e.g. 2-naphthyl), indenyl, fenanthryl or anthracyl radical. The aryl radical may be optionally substituted by one or more substituents such as hydroxy, mercapto, halo, alkyl, phenyl, alkoxy, haloalkyl, nitro, cyano, dialkylamino, aminoalkyl, acyl, alkoxycarbonyl, etc.

“Heterocyclyl” refers to a stable, typically 3- to 18-membered, ring radical which consists of carbon atoms and from one to five heteroatoms selected from the group consisting of nitrogen, oxygen, and sulfur, preferably a 4- to 15-membered ring with one or more heteroatoms, preferably a 4- to 8-membered ring with one or more heteroatoms, more preferably a 5- or 6-membered ring with one or more heteroatoms. It may be aromatic or not aromatic. For the purposes of this invention, the heterocycle may be a monocyclic, bicyclic or tricyclic ring system, which may include fused ring systems; and the nitrogen, carbon or sulfur atoms in the heterocyclyl radical may be optionally oxidized; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran, coumarine, morpholine; pyrrole, pyrazole, oxazole, isoxazole, triazole, imidazole, etc.

“Alkoxy” refers to a radical of the formula —ORₐ where Rₐ is an alkyl radical as defined above having one or more (e.g., 1, 2, 3 or 4) oxygen linkages and typically from 1 to about 12, 1 to about 8 or 1 to about 6 carbon atoms, e. g., methoxy, ethoxy, propoxy, etc.

“Aryloxy” refers to a radical of formula —O-aryl, where aryl is as previously defined. Some examples of aryloxy compounds are —O-phenyl (i.e. phenoxy), —O-p-tolyl, —O-m-tolyl, —O-o-tolyl or —O-naphthyl.

“Amino” refers to a radical of the formula —NH₂, —NHRₐ or —NRₐR_{b}, optionally quaternized. In an embodiment of the invention each of Rₐ and R_{b} is independently selected from hydrogen and an alkyl radical as defined above. Therefore, examples of amino groups are, methylamino, ethylamino, dimethylamino, diethylamino, propylamino, etc.

“Halogen”, “halo” or “hal” refers to bromo, chloro, iodo or fluoro.

“Fused ring system” refers to a polycyclic ring system that contains fused rings. Typically, the fused ring system contains 2 or 3 rings and/or up to 18 ring atoms. As defined above, cycloalkyl radicals, aryl radicals and heterocyclyl radicals may form fused ring systems. Thus, fused ring system may be aromatic, partially aromatic or not aromatic and may contain heteroatoms. A spiro ring system is not a fused-polycyclic by this definition, but fused polycyclic ring systems of the invention may themselves have spiro rings attached thereto via a single ring atom of the system. Examples of fused ring systems are, but are not limited to, adamantyl, naphthyl (e.g. 2-naphthyl), indenyl, fenanthryl, anthracyl, pyrenyl, benzimidazole, benzothiazole, etc.

Unless otherwise stated specifically in the specification, all the groups may be optionally substituted, if applicable. References herein to substituted groups in the compounds of the present invention refer to the specified moiety that may be substituted at one or more (e.g., 1, 2, 3 or 4) available positions by one or more suitable groups, e. g., halogen such as fluoro, chloro, bromo and iodo; cyano; hydroxyl; nitro; azido; acyl, such as alkanoyl, e.g. a C₁₋₆ alkanoyl group, and the like; carboxamido; alkyl groups including those groups having 1 to about 12 carbon atoms or from 1 to about 6 carbon atoms and more preferably 1-3 carbon atoms; alkenyl and alkynyl groups including groups having one or more (e.g., 1, 2, 3 or 4) unsaturated linkages and from 2 to about 12 carbon or from 2 to about 6 carbon atoms; alkoxy groups having one or more (e.g., 1, 2, 3 or 4) oxygen linkages and from 1 to about 12 carbon atoms or 1 to about 6 carbon atoms; aryloxy such as phenoxy; alkylthio groups including those moieties having one or more (e.g., 1, 2, 3 or 4) thioether linkages and from 1 to about 12 carbon atoms or from 1 to about 6 carbon atoms; alkylsulfinyl groups including those moieties having one or more (e.g., 1, 2, 3 or 4) sulfinyl linkages and from 1 to about 12 carbon atoms or from 1 to about 6 carbon atoms; alkylsulfonyl groups including those moieties having one or more (e.g., 1, 2, 3 or 4) sulfonyl linkages and from 1 to about 12 carbon atoms or from 1 to about 6 carbon atoms; aminoalkyl groups such as groups having one or more (e.g., 1, 2, 3 or 4) N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

The term “salt” must be understood as any form of a compound used in accordance with this invention in which said compound is in ionic form or is charged and coupled to a counter-ion (a cation or anion) or is in solution. This definition also includes quaternary ammonium salts and complexes of the molecule with other molecules and ions, particularly, complexes formed via ionic interactions. The definition includes in particular physiologically acceptable salts; this term must be understood as equivalent to “pharmacologically acceptable salts” or “pharmaceutically acceptable salts”.

The term “pharmaceutically acceptable salts” in the context of this invention means any salt that is tolerated physiologically (normally meaning that it is not toxic, particularly, as a result of the counter-ion) when used in an appropriate manner for a treatment, applied or used, particularly, in humans and/or mammals. These physiologically acceptable salts may be formed with cations or bases and, in the context of this invention, are understood to be salts formed by at least one compound used in accordance with the invention—normally an acid (deprotonated)—such as an anion and at least one physiologically tolerated cation, preferably inorganic, particularly when used on humans and/or mammals. Salts with alkali and alkali earth metals are preferred particularly, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” in accordance with this invention should be understood as meaning any form of a compound in accordance with the invention in which said compound is bonded by a non-covalent bond to another molecule (normally a polar solvent), including especially hydrates and alcoholates, like for example, methanolate. A preferred solvate is the hydrate.

Any compound that is a prodrug of a compound referred to herein is also within the scope of the invention. The term “prodrug” is used in its broadest sense and encompasses those derivatives that are converted in vivo to the compounds of the invention. Examples of prodrugs include, but are not limited to, derivatives of the compounds referred to herein such as compounds of formula (I) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6th ed. (Donald J. Abraham ed., 2001, Wiley) and “Design and Applications of Prodrugs” (H. Bundgaard ed., 1985, Harwood Academic Publishers).

Any compound referred to herein is intended to represent such specific compound as well as certain variations or forms. In particular, compounds referred to herein may have asymmetric centres and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are enamine-imine, amide-imidic acid, keto-enol, lactam-lactim, etc.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of the invention or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrug.

As used herein, the terms “treat”, “treating” and “treatment” include the eradication, removal, reversion, alleviation, modification, or control of pain after its onset.

As used herein, the terms “prevention”, “preventing”, “preventive” “prevent” and “prophylaxis” refer to the capacity of a therapeutic to avoid, minimize or difficult the onset or development of a disease or condition before its onset, in this case pain.

Therefore, by “treating” or “treatment” and/or “preventing” or “prevention”, as a whole, is meant at least a suppression or an amelioration of the symptoms associated with the condition afflicting the subject, where suppression and amelioration are used in a broad sense to refer to at least a reduction in the magnitude of a parameter, e.g., symptom associated with the condition being treated, such as pain. As such, the method of the present invention also includes situations where the condition is completely inhibited, e.g., prevented from happening, or stopped, e.g., terminated, such that the subject no longer experiences the condition. As such, the present method includes both preventing and managing pain, particularly, peripheral neuropathic pain, allodynia, causalgia, hyperalgesia, hyperesthesia, hyperpathia, neuralgia, neuritis or neuropathy. In a preferred embodiment of the present invention, pain refers specifically to “post-operative pain”.

As used herein, the term “potentiating the analgesic effect of a NSAID” refers to the increase in the effectiveness of the analgesic effect of said NSAID produced by sigma ligands. In an embodiment of the invention, said potentiating effect induces an increase in the analgesic effect of the NSAID by a factor of 1.2, 1.5, 2, 3, 4 or more when compared with the NSAID when administered in isolation. The measurement can be done following any known method in the art.

As used herein, the term “potentiating the analgesic effect of a Sigma ligand” refers to the increase in the effectiveness of the analgesic effect of said Sigma ligand produced by NSAID. In an embodiment of the invention said potentiating effect induces an increase in the analgesic effect of the Sigma ligand by a factor of 1.2, 1.5, 2, 3, 4 or more when compared with the Sigma ligand when administered in isolation. The measurement can be done following any known method in the art.

As above mentioned, the Sigma ligands, such as those of general formula (I), surprisingly potentiate the analgesic effect of NSAIDs, thus reducing the doses needed to obtain effective analgesia of the latter.

“Synergy” may be defined as the interaction of multiple elements in a system to produce an effect different from or greater than the sum of their individual effects. Thus, the combinations of the present invention are synergistic.

“The sigma receptor/s” as used in this application is/are well known and defined using the following citation: “this binding site represents a typical protein different from opioid, NMDA, dopaminergic, and other known neurotransmitter or hormone receptor families” (Ronsisvalle, G. et al., 2001). Pharmacological data based on ligand binding studies, anatomical distribution and biochemical features distinguish at least two subtypes of σ receptors (Quiron R. et al., 1992; Leitner M. L., 1994; Hellewell, S. B. and Bowen, W. D., 1990; Ronsisvalle, G. et al., 2001). The protein sequences of the sigma receptors (Sigma 1 (σ1) and Sigma 2 (σ2)) are known in the art (e.g. Prasad, P. D. et al., 1998). They show a very high affinity to various analgesics (e.g. pentazocine).

As used herein, the terms “Sigma ligand” or “Sigma receptor ligand” refer to any “compound binding to the Sigma receptor”. Compounds binding to the sigma receptor are well known in the art. “Compound/s binding to the Sigma receptor” or “sigma ligand” as used in this application is/are preferably defined as a compound having an IC₅₀ value of ≦5000 nM, more preferably ≦1000 nM, more preferably ≦500 nM on the sigma receptor. More preferably, the IC₅₀ value is ≦250 nM. More preferably, the IC₅₀ value is ≦100 nM. Most preferably, the IC₅₀ value is ≦50 nM. The half maximal inhibitory concentration (IC₅₀) is a measure of the effectiveness of a compound in inhibiting biological or biochemical function. The IC₅₀ is the concentration of competing ligand which displaces 50% of the specific binding of the radioligand. Additionally, the wording “Compound/s binding to the sigma receptor”, as used in the present application is preferably defined as having at least ≧50% displacement using 10 nM radioligand specific for the sigma receptor (e.g. preferably [³H]-(+) pentazocine) whereby the sigma receptor may be any sigma receptor subtype. Preferably, said compounds bind to the sigma-1 receptor subtype.

Further, said compounds binding to the sigma receptor as defined herein, may be antagonists, inverse agonists, agonists, partial antagonists and/or partial agonists. The sigma ligand according to the present invention is preferably a sigma receptor antagonist in the form of a (neutral) antagonist, an inverse agonist or a partial antagonist.

In a preferred embodiment of the invention the Sigma receptor ligand is a selective Sigma-1 antagonist, preferably in the form of a (neutral) antagonist, an inverse agonist or a partial antagonist, more preferably a selective Sigma-1 (neutral) antagonist.

An “agonist” is defined as a compound that binds to a receptor and has an intrinsic effect, and thus, increases the basal activity of a receptor when it contacts the receptor.

An “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding.

A “partial antagonist” is defined as a compound that binds to the receptor and generates an antagonist response; however, a partial antagonist does not generate the full antagonist response. Partial antagonists are weak antagonists, thereby blocking partially the action of an agonist or inverse agonist on the receptor.

An “inverse agonist” is defined as a compound that produces an effect opposite to that of the agonist by occupying the same receptor and, thus, decreases the basal activity of a receptor (i.e., signalling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist.

Table 1 lists some sigma ligands known in the art (i.e. having an IC₅₀≦5000 nM). Some of these compounds may bind to the sigma-1 and/or to the sigma-2 receptor. These sigma ligands also include their respective salts, bases, and acids.

TABLE 1

| | |
|---|---|
| Acetophenazine Maleate | Fluphenazine Decanoate DiHCl |
| Alverine | Fluphenazine Enanthate DiHCl |
| Aminobenztropine | Fluphenazine HCl |
| Amorolfine HCl | Fluphenazine N-Mustard DiHCl |
| AN2/AVex-73; AE-37; | Fluspidine |
| ANAVEX 2-73; N- | Fentanyl |
| (2,2-Diphenyltetrahydrofuran- | GBR-12935 DiHCl |
| 3-ylmethyl)- | HEAT HCl |
| N,N-dimethylamine | I-693, 403 |
| Anileridine | Ifenprodil Tartrate |
| BD-1063 | Igmesine |
| BD-1008 | LR132 |
| BD-1047 | Lobeline HCl |
| Benproperine Phosphate | Lomerizine diHCl |
| Benztropine Mesylate | Loperamide |
| Bromhexine HCl | Mebeverine |
| Bromperidol | Naftifine |
| Carbetapentane Citrate | NE-100 |
| Cinnarizine | Opipramol |
| Cis(Z)-Flupentixol DiHCl | Oxybutynin |
| Clobenztropine | Pirlindole |
| Clorgyline HCl | Perphenazine |
| Cutamesine hydrochloride | Sertraline |
| Cyclobenzaprine HCl | Sufentanyl |
| Dicyclomine HCl | Terbinafine HCl |
| Dimemorphan | Trifluoperazine HCl |
| Dextromethorphan | Trifluperidol HCl |
| Ditolylguanidine | Trimeprazine Hemi-L-Tartrate |
| Duloxetine | Vanoxerine |
| Dibenzheptoprine | Xylazine |
| Donepezil | |
| Eliprodil | |
| Fluvoxamine | |
| Flunarizine diHCl | |

Preferably, the table above includes also haloperidol, haloperidol metabolite I (4-(4-chlorophenyl)-4-hydroxypiperidine) and haloperidol metabolite II (4-(4-chlorophenyl)-□-(4-fluorophenyl)-4-hydroxy-1-piperidinebutanol) also called reduced haloperidol Studies performed in rodent brain membranes and human neuroblastoma cells showed that metabolites I and II of haloperidol bind to σ1 receptors with less affinity than haloperidol, but show much lower (metabolite II) or no affinity (metabolite I) for D2 receptors. Reduced haloperidol or metabolite II, an active metabolite of haloperidol that is produced in humans, shows a high affinity (in the low nanomolar range) for sigma-1 receptors, and produces an irreversible blockade of sigma-1 receptors both in experimental animals and human cells.

In a preferred embodiment, the Sigma receptor ligand in the context of the present invention has the general formula (I) as depicted above.

In a preferred embodiment, R₁ in the compounds of general formula (I) is selected from H, —COR₈, and substituted or unsubstituted alkyl. More preferably, R₁ is selected from H, methyl and acetyl. A more preferred embodiment is when R₁ is H.

In another preferred embodiment, R₂ in the compounds of formula (I) represents H or substituted or unsubstituted alkyl, more preferably methyl.

In a particular embodiment of the invention, R₃ and R₄ in the compounds of formula (I) are situated in the meta and para positions of the phenyl group, and preferably, they are selected independently from halogen and substituted or unsubstituted alkyl.

In an especially preferred embodiment of the invention, in the compounds of formula (I) both R₃ and R₄ together with the phenyl group form an optionally substituted fused ring system. More preferably, said fused ring system is selected from a substituted or unsubstituted fused aryl group and a substituted or unsubstituted aromatic or partially aromatic fused heterocyclyl group. Said fused ring system preferably contains two rings and/or from 9 to about 18 ring atoms, more preferably 9 or 10 ring atoms. Even more preferably, the fused ring system is naphthyl, especially a 2-naphthyl ring system, substituted or unsubstituted.

Also in the compounds of formula (I), embodiments where n is selected from 2, 3 or 4 are preferred in the context of the present invention, more preferably n is 2.

In another embodiment it is preferred in the compounds of formula (I) that R₅ and R₆ are, each independently, C₁₋₆alkyl, or together with the nitrogen atom to which they are attached form a substituted or unsubstituted heterocyclyl group, in particular a group chosen among morpholinyl, piperidinyl, and pyrrolidinyl group. More preferably, R₅ and R₆ together form a morpholine-4-yl group.

In additional preferred embodiments, the preferences described above for the different substituents are combined. The present invention is also directed to such combinations of preferred substitutions in the formula (I) above.

In preferred variants of the invention, the Sigma ligand of general formula (I) is selected from:
[1] 4-{2-(1-(3,4-dichlorophenyl)-5-methyl-1H pyrazol-3-yloxy)ethyl} morpholine,
[2] 2-[1-(3,4-Dichlorophenyl)-5-methyl-1H-pyrazol-3-yloxy]-N,N-diethylethanamine,
[3] 1-(3,4-Dichlorophenyl)-5-methyl-3-[2-(pyrrolidin-1-yl)ethoxy]-1H-pyrazole,
[4] 1-(3,4-Dichlorophenyl)-5-methyl-3-[3-(pyrrolidin-1-yl)propoxy]-1H-pyrazole,
[5] 1-{2-[1-(3,4-Dichlorophenyl)-5-methyl-1H-pyrazol-3-yloxy]ethyl}piperidine,
[6] 1-{2-[1-(3,4-dichlorophenyl)-5-methyl-1H-pyrazol-3-yloxy]ethyl}-1H-imidazole,
[7] 3-{1-[2-(1-(3,4-Dichlorophenyl)-5-methyl-1H-pyrazol-3-yloxy)ethyl]piperidin-4-yl}-3H-imidazo[4,5-b]pyridine,
[8] 1-{2-[1-(3,4-Dichlorophenyl)-5-methyl-1H-pyrazol-3-yloxy]ethyl}-4-methylpiperazine,
[9] Ethyl 4-{2-[1-(3,4-dichlorophenyl)-5-methyl-1H-pyrazol-3-yloxy]ethyl}piperazine carboxylate,
[10] 1-(4-(2-(1-(3,4-dichlorophenyl)-5-methyl-1H-pyrazol-3-yloxy)ethyl)piperazin-1-yl)ethanone,
[11] 4-{2-[1-(4-Methoxyphenyl)-5-methyl-1H-pyrazol-3-yloxy]ethyl}morpholine,
[12] 1-(4-Methoxyphenyl)-5-methyl-3-[2-(pyrrolidin-1-yl)ethoxy]-1H-pyrazole,
[13] 1-(4-Methoxyphenyl)-5-methyl-3-[3-(pyrrolidin-1-yl)propoxy]-1H-pyrazole,
[14] 1-[2-(1-(4-Methoxyphenyl)-5-methyl-1H-pyrazol-3-yloxy)ethyl]piperidine,
[15] 1-{2-[1-(4-Methoxyphenyl)-5-methyl-1H-pyrazol-3-yloxy]ethyl}-1H-imidazole,
[16] 4-{2-[1-(3,4-Dichlorophenyl)-5-phenyl-1H-pyrazol-3-yloxy]ethyl}morpholine,
[17] 1-(3,4-Dichlorophenyl)-5-phenyl-3-[2-(pyrrolidin-1-yl)ethoxy]-1H-pyrazole,
[18] 1-(3,4-Dichlorophenyl)-5-phenyl-3-[3-(pyrrolidin-1-yl)propoxy]-1H-pyrazole,
[19] 1-{2-[1-(3,4-Dichlorophenyl)-5-phenyl-1H-pyrazol-3-yloxy]ethyl}piperidine,
[20] 1-{2-[1-(3,4-Dichlorophenyl)-5-phenyl-1H-pyrazol-3-yloxy]ethyl}-1H-imidazole,
[21] 2-{2-[1-(3,4-dichlorophenyl)-5-phenyl-1H-pyrazol-3-yloxy]ethyl}-1,2,3,4-tetrahydroisoquinoline,
[22] 4-{4-[1-(3,4-Dichlorophenyl)-5-methyl-1H-pyrazol-3-yloxy]butyl}morpholine,
[23] 1-(3,4-Dichlorophenyl)-5-methyl-3-[4-(pyrrolidin-1-yl)butoxy]-1H-pyrazole,
[24] 1-{4-[1-(3,4-Dichlorophenyl)-5-methyl-1H-pyrazol-3-yloxy]butyl}piperidine,
[25] 1-{4-[1-(3,4-Dichlorophenyl)-5-methyl-1H-pyrazol-3-yloxy]butyl}-4-methylpiperazine,
[26] 1-{4-[1-(3,4-Dichlorophenyl)-5-methyl-1H-pyrazol-3-yloxy]butyl}-1H-imidazole,
[27] 4-[1-(3,4-Dichlorophenyl)-5-methyl-1H-pyrazol-3-yloxy]-N,N-diethylbutan-1-amine,
[28] 1-{4-[1-(3,4-dichlorophenyl)-5-methyl-1H-pyrazol-3-yloxy]butyl}-4-phenylpiperidine,
[29] 1-{4-[1-(3,4-dichlorophenyl)-5-methyl-1H-pyrazol-3-yloxy]butyl}-6,7-dihydro-1H-indol-4(5H)-one,
[30] 2-{4-[1-(3,4-dichlorophenyl)-5-methyl-1H-pyrazol-3-yloxy]butyl}-1,2,3,4-tetrahydroisoquinoline,
[31] 4-{2-[1-(3,4-dichlorophenyl)-5-isopropyl-1H-pyrazol-3-yloxy]ethyl}morpholine,
[32] 2-[1-(3,4-Dichlorophenyl)-5-isopropyl-1H-pyrazol-3-yloxy]-N,N-diethylethanamine,
[33] 1-(3,4-Dichlorophenyl)-5-isopropyl-3-[2-(pyrrolidin-1-yl)ethoxy]-1H-pyrazole,
[34] 1-(3,4-Dichlorophenyl)-5-isopropyl-3-[3-(pyrrolidin-1-yl)propoxy]-1H-pyrazole,
[35] 1-{2-[1-(3,4-Dichlorophenyl)-5-isopropyl-1H-pyrazol-3-yloxy]ethyl}piperidine,
[36] 2-{2-[1-(3,4-dichlorophenyl)-5-isopropyl-1H-pyrazol-3-yloxy]ethyl}-1,2,3,4-tetrahydroisoquinoline,
[37] 4-{2-[1-(3,4-dichlorophenyl)-1H-pyrazol-3-yloxy]ethyl}morpholine,
[38] 2-[1-(3,4-dichlorophenyl)-1H-pyrazol-3-yloxy] N,N-diethylethanamine,
[39] 1-(3,4-dichlorophenyl)-3-[2-(pyrrolidin-1-yl)ethoxy]-1H-pyrazole,
[40] 1-{2-[1-(3,4-dichlorophenyl)-1H-pyrazol-3-yloxy]ethyl}piperidine,
[41] 1-(3,4-dichlorophenyl)-3-[3-(pyrrolidin-1-yl)propoxy]-1H-pyrazole,
[42] 1-{2-[1-(3,4-Dichlorophenyl)-5-methyl-1H-pyrazol-3-yloxy]ethyl}piperazine,
[43] 1-{2-[1-(3,4-Dichlorophenyl)-5-methyl-1H-pyrazol-3-yloxy]ethyl}pyrrolidin-3-amine,
[44] 4-{2-[1-(3,4-Dichlorophenyl)-4,5-dimethyl-1H-pyrazol-3-yloxy]ethyl}morpholine,
[46] 2-[1-(3,4-Dichlorophenyl)-4,5-dimethyl-1H-pyrazol-3-yloxy]-N,N-diethylethanamine,
[47] 1-(3,4-Dichlorophenyl)-4,5-dimethyl-3-[2-(pyrrolidin-1-Methoxy]-1H-pyrazole,
[48] 1-(3,4-Dichlorophenyl)-4,5-dimethyl-3-[3-(pyrrolidin-1-yl)propoxy]-1H-pyrazole,
[49] 1-{2-[1-(3,4-Dichlorophenyl)-4,5-dimethyl-1H-pyrazol-3-yloxy]ethyl}piperidine,
[50] 4-{4-[1-(3,4-dichlorophenyl)-1H-pyrazol-3-yloxy]butyl}morpholine,
[51] (2S,6R)-4-{4-[1-(3,4-dichlorophenyl)-1H-pyrazol-3-yloxy]butyl}-2,6-dimethylmorpholine,
[52] 1-{4-[1-(3,4-Dichlorophenyl)-1H-pyrazol-3-yloxy]butyl}piperidine,
[53] 1-(3,4-Dichlorophenyl)-3-[4-(pyrrolidin-1-yl)butoxy]-1H-pyrazole,
[55] 4-[1-(3,4-dichlorophenyl)-1H-pyrazol-3-yloxy]-N,N-diethylbutan-1-amine,
[56] N-benzyl-4-[1-(3,4-dichlorophenyl)-1H-pyrazol-3-yloxy]-N-methylbutan-1-amine,
[57] 4-[1-(3,4-dichlorophenyl)-1H-pyrazol-3-yloxy]-N-(2-methoxyethyl)-N-methylbutan-1-amine,
[58] 4-{4-[1-(3,4-dichlorophenyl)-1H-pyrazol-3-yloxy]butyl}thiomorpholine,
[59] 1-[1-(3,4-Dichlorophenyl)-5-methyl-3-(2-morpholinoethoxy)-1H-pyrazol-4-yl]ethanone,
[60] 1-{1-(3,4-dichlorophenyl)-5-methyl-3-[2-(pyrrolidin-1-Methoxy]-1H-pyrazol-4-yl}ethanone,
[61] 1-{1-(3,4-dichlorophenyl)-5-methyl-3-[2-(piperidin-1-yl)ethoxy]-1H-pyrazol-4-yl}ethanone,
[62] 1-{1-(3,4-dichlorophenyl)-3-[2-(diethylamino)ethoxy]-5-methyl-1H-pyrazol-4-yl}ethanone,
[63] 4-{2-[5-Methyl-1-(naphthalen-2-yl)-1H-pyrazol-3-yloxy]ethyl}morpholine,
[64] N,N-Diethyl-2-[5-methyl-1-(naphthalen-2-yl)-1H-pyrazol-3-yloxy] ethanamine,
[65] 1-{2-[5-Methyl-1-(naphthalen-2-yl)-1H-pyrazol-3-yloxy]ethyl}piperidine, and
[66] 5-Methyl-1-(naphthalen-2-yl)-3-[2-(pyrrolidin-1-yl)ethoxy]-1H-pyrazole,
   or their pharmaceutically acceptable salts, solvates or prodrugs.

In a preferred variant of the invention, the Sigma ligand of general formula (I) is 4-{2-[5-Methyl-1-(naphthalen-2-yl)-1H-pyrazol-3-yloxy]ethyl}morpholine or a salt thereof.

Preferably, the compound of general formula I used is 4-{2-[5-Methyl-1-(naphthalen-2-yl)-1H-pyrazol-3-yloxy]ethyl}morpholine hydrochloride.

These particular compounds are designated in the examples of the present invention as compound 63 and compound 63.HCl.

The compounds of general formula (I) and their salts or solvates can be prepared as disclosed in the previous application WO2006/021462.

In other embodiments, the combination of the invention comprises BD1063 as the Sigma ligand.

NSAIDs are anti-inflammatory agents which are believed to act by disrupting the arachidonic acid cascade. Some of them are antipyretics (drugs that reduce fever) in addition to having analgesic and anti-inflammatory actions. As commented above the NSAIDs block the cyclooxygenase enzyme that catalyzes the conversion of arachidonic acid to the prostaglandins PGG₂ and PGH₂. Since these two cyclic endoperoxides are the precursors of all other prostaglandins, the implications of cyclooxygenase inhibition are significant. Prostaglandin E₁ is known to be a potent pyrogen (fever-causing agent), and PGE₂ causes pain, edema, erythema (reddening of the skin), and fever. The prostaglandin endoperoxides (PGG₂ and PGH₂) can also produce pain, and inhibition of their synthesis can thus account for the action of the nonsteroidal anti-inflammatory agents (Medicinal Chemistry-A Molecular and Biochemical Approach; third edition; Thomas Nogrady; Donald F. Weaver; Oxford University Press 2005).

The NSAIDs may be classified as follows (this list provides non-limiting examples for each category):

1. NSAIDs: Non-selective cyclo-oxygenase inhibitors
a. Arylanthranilic acids (mefenamic acid, meclofenamate)
b. Arylbutyric acids (nabumetone)
c. Arylpropionic acids (ibuprofen, dexibuprofen, ketoprofen, fenoprofen, naproxen, ketorolac, dexketoprofen, flurbiprofen, oxaprozin, loxoprofen)
d. Indene derivatives (sulindac)
e. Indole derivatives (indomethacin)
f. Naphthylacetic acid derivatives (nabumetone)
g. Oxicams (piroxicam, meloxicam, tenoxicam)
h. Phenylacetic acid derivatives (diclofenac)
i. Phenylalkanoic acid derivatives (flurbiprofen)
j. Pyrazolone derivatives (phenylbutazone, azapropazone, metamizole)
k. Pyrrolealkanoic acid derivatives (tolmetin)
l. Salicylate derivatives (aspirin, diflunisal, salsalate)

2. NSAIDs: Selective COX-2 inhibitors
a. Coxibs (celecoxib, rofecoxib)

3. NSAIDs: Paracetamol or Acetaminophen

Another possible classification is as follows (this list provides non-limiting examples for each category):
Pyrazolidines: ampyrone, azapropazone, clofezone, kebuzone, metamizole, mofebutazone, nifenazone, oxyphenbutazone, phenazone, phenylbutazone, sulfinpyrazone, suxibuzone, feprazone.
Salicylates: aspirin (acetylsalicylic acid), aloxiprin, benorylate, carbasalate calcium, diflunisal, dipyrocetyl, ethenzamide, guacetisal, magnesium salicylate, methyl salicylate, salsalate, salicin, salicylamide, sodium salicylate.
Acetic acid derivatives and related substances: aceclofenac, acemetacin, alclofenac, amfenac, bendazac, bromfenac, bumadizone, bufexamac, diclofenac, difenpiramide, etodolac, felbinac, fentiazac, indomethacin, farnesil, ketorolac, lonazolac, oxametacin, proglumetacin, sulindac, tolmetin, zomepirac.
Oxicams: ampiroxicam, droxicam, lornoxicam, meloxicam, piroxicam, tenoxicam.
Propionic acid derivatives (profens): alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, loxoprofen, miroprofen, naproxen, oxaprozin, pirprofen, suprofen, tarenflurbil, tepoxalin, tiaprofenic acid, vedaprofen, cox-inhibiting nitric oxide donator: naproxcinod
N-Arylanthranilic acids (fenamates): azapropazone, etofenamate, flufenamic acid, flunixin, meclofenamic acid, meclofenamate, mefenamic acid, morniflumate, niflumic acid, tolfenamic acid.
Coxibs: celecoxib, cimicoxib, deracoxib, etoricoxib, firocoxib, lumiracoxib, mavacoxib, parecoxib, robenacoxib, rofecoxib, valdecoxib.
Paracetamol or acetaminophen
Other: aminopropionitrile, benzydamine, chondroitin sulfate, diacerein, fluproquazone, glucosamine, glycosaminoglycan, magnesium salicylate, nabumetone, nimesulide, oxaceprol, proquazone, superoxide dismutase/orgotein, tenidap.

All the NSAIDs mentioned in the above classifications are contemplated in the present invention.

Therefore, as used herein, the term “NSAID” refers to drugs with analgesic and anti-inflammatory effects which additionally can show antipyretic (fever-reducing) activity.

The term “NSAID” includes, but is not limited to, those agents which inhibit cyclooxygenase, the enzyme responsible for the biosynthesis of the prostaglandins and certain autocoid inhibitors, including inhibitors of the various isoenzymes of cyclooxygenase (including, but not limited to, cyclooxygenase-1, -2 and/or putative COX-3).

In particular the term “NSAID” includes, but is not limited to, aceclofenac, acemetacin, acetaminophen, acetaminosalol, acetyl-salicylic acid, acetyl-salicylic-2-amino-4-picoline-acid, 5-aminoacetylsalicylic acid, alclofenac, aloxiprin, alminoprofen, aminoprofen, amfenac, ampyrone, ampiroxicam, anileridine, azapropazone, bendazac, benorylate, benoxaprofen, bermoprofen, abisabolol, bromfenac, 5-bromosalicylic acid acetate, bromosaligenin, bucloxic acid, bufexamac, butibufen, bumadizone, carbasalate, carprofen, celecoxib, chromoglycate, cimicoxib, cinmetacin, clindanac, clofezone, clopirac, deracoxib, dexibuprofen, dexketoprofen, particularly sodium dexketoprofen, diclofenac, difenpiramide, diflunisal, ditazol, dipyrocetyl, droxicam, enfenamic acid, ethenzamide, etodolac, etofenamate, etoricoxib, farnesil, felbinac, fenbufen, fenclozic acid, fendosal, fenoprofen, fentiazac, fepradinol, feprazone, firocoxib, flufenac, flufenamic acid, flunixin, flunoxaprofen, flurbiprofen, glutametacin, glycol salicylate, guacetisal, ibufenac, ibuprofen, ibuproxam, indomethacin, indoprofen, isofezolac, isoxepac, isoxicam, ketoprofen, ketorolac, kebuzone, lonazolac, lornoxicam, loxoprofen, lumiracoxib, mavacoxib, magnesium salicylate, meclofenamate, meclofenamic acid, mefenamic acid, meloxicam, mesalamine, metamizole, methyl salicylate, metiazinic acid, miroprofen, mofezolac, mofebutazone, montelukast, morniflumate, mycophenolic acid, nabumetone, naproxcinod, naproxen, nifenazone, niflumic acid, nimesulide, olsalazine, oxaceprol, oxametacin, oxaprozin, oxyphenbutazone, paracetamol, parecoxib, parsalmide, perisoxal, phenazone, phenyl-acethyl-salicylate, phenylbutazone, phenylsalicylate, pyrazolac, piroxicam, pirprofen, pranoprofen, proglumetacin, proquazone, protizinic acid, reserveratol, robenacoxib, rofecoxib, salacetamide, salicylamide, salicylamide-O-acetyl acid, salicylsulphuric acid, salicin, salicylamide, salsalate, sodium salicylate, sulfinpyrazone, sulindac, suprofen, suxibutazone, tamoxifen, tenidap, tenoxicam, theophylline, tiaprofenic acid, tiaramide, ticlopridine, tinoridine, tolfenamic acid, tolmetin, tropesin, valdecoxib, vedaprofen, xenbucin, ximoprofen, zaltoprofen, zomepirac, tomoxiprol, zafirlukast, cyclosporine, derivatives, salts and mixtures thereof.

In one embodiment, the NSAID is selected from the group of NSAIDs consisting of pyrazolidines, salicylates, acetic acid derivatives, oxicams, propionic acid derivative, N-arylanthranilic acids, paracetamol and coxibs. In a preferred embodiment the NSAID is selected from the group of NSAIDs consisting of pyrazolidines, acetic acid derivatives, paracetamol and coxibs.

Preferred NSAIDs are selected from the groups consisting of paracetamol, ibuprofen, naproxen, ketoprofen, dexketoprofen, mefenamic acid, piroxicam, meloxicam, flurbiprofen, aceclofenac, acemetacin, alclofenac, amfenac, bendazac, bromfenac, bumadizone, bufexamac, diclofenac, difenpiramide, etodolac, felbinac, fentiazac, indomethacin, ketorolac, lonazolac, oxametacin, proglumetacin, sulindac, tolmetin, zomepirac, celecoxib, cimicoxib, deracoxib, etoricoxib, firocoxib, lumiracoxib, mavacoxib, parecoxib, robenacoxib, rofecoxib, valdecoxib, ampyrone, azapropazone, clofezone, kebuzone, metamizole, mofebutazone, nifenazone, oxyphenbutazone, phenazone, phenylbutazone, sulfinpyrazone, suxibuzone and feprazone.

More preferred NSAIDs are selected from the group consisting of paracetamol, ibuprofen, naproxen, diclofenac, celecoxib and metamizole. In a particular embodiment the NSAID is paracetamol. In another particular embodiment the NSAID is selected from the group consisting of ibuprofen, naproxen, diclofenac, celecoxib and metamizole.

A particular embodiment refers to the combination of the invention comprising 4-{2-[5-methyl-1-(naphthalen-2-yl)-1H-pyrazol-3-yloxy]ethyl}morpholine or a pharmaceutically acceptable salt, isomer, prodrug or solvate thereof and a NSAID selected from the group consisting of paracetamol, ibuprofen, naproxen, diclofenac, celecoxib and metamizole.

A particular embodiment refers to the combination of the invention comprising 4-{2-[5-methyl-1-(naphthalen-2-yl)-1H-pyrazol-3-yloxy]ethyl}morpholine or a salt thereof and diclofenac.

A particular embodiment refers to the combination of the invention comprising 4-{2-[5-methyl-1-(naphthalen-2-yl)-1H-pyrazol-3-yloxy]ethyl}morpholine hydrochloride and diclofenac.

A particular embodiment refers to the combination of the invention comprising 4-{2-[5-methyl-1-(naphthalen-2-yl)-1H-pyrazol-3-yloxy]ethyl}morpholine or a salt thereof and celecoxib.

A particular embodiment refers to the combination of the invention comprising 4-{2-[5-methyl-1-(naphthalen-2-yl)-1H-pyrazol-3-yloxy]ethyl}morpholine hydrochloride and celecoxib.

A particular embodiment refers to the combination of the invention comprising 4-{2-[5-methyl-1-(naphthalen-2-yl)-1H-pyrazol-3-yloxy]ethyl}morpholine or a salt thereof and metamizole.

A particular embodiment refers to the combination of the invention comprising 4-{2-[5-methyl-1-(naphthalen-2-yl)-1H-pyrazol-3-yloxy]ethyl}morpholine hydrochloride and metamizole.

A particular embodiment refers to the combination of the invention comprising 4-{2-[5-methyl-1-(naphthalen-2-yl)-1H-pyrazol-3-yloxy]ethyl}morpholine or a salt thereof and ibuprofen.

A particular embodiment refers to the combination of the invention comprising 4-{2-[5-methyl-1-(naphthalen-2-yl)-1H-pyrazol-3-yloxy]ethyl}morpholine hydrochloride and ibuprofen.

A particular embodiment refers to the combination of the invention comprising 4-{2-[5-methyl-1-(naphthalen-2-yl)-1H-pyrazol-3-yloxy]ethyl}morpholine or a salt thereof and naproxen.

A particular embodiment refers to the combination of the invention comprising 4-{2-[5-methyl-1-(naphthalen-2-yl)-1H-pyrazol-3-yloxy]ethyl}morpholine hydrochloride and naproxen.

A particular embodiment refers to the combination of the invention comprising 4-{2-[5-methyl-1-(naphthalen-2-yl)-1H-pyrazol-3-yloxy]ethyl}morpholine or a salt thereof and paracetamol.

A particular embodiment refers to the combination of the invention comprising 4-{2-[5-methyl-1-(naphthalen-2-yl)-1H-pyrazol-3-yloxy]ethyl}morpholine hydrochloride and paracetamol.

In other embodiments, the combination of the invention comprises BD1063 and a NSAID selected from the group consisting of paracetamol, diclofenac, celecoxib and metamizole.

The present invention refers also to the use of medicaments or pharmaceutical compositions comprising at least one Sigma ligand of general formula (I) as defined above, or a pharmaceutically acceptable salt, isomer, prodrug or solvate thereof, and at least one NSAID combined jointly or separately, together with at least a pharmaceutically acceptable excipient.

The term “excipient” refers to components of a drug compound other than the active ingredient (definition obtained from the European Medicines Agency—EMA). They preferably include a “carrier, adjuvant and/or vehicle”. Carriers are forms to which substances are incorporated to improve the delivery and the effectiveness of drugs. Drug carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions (U.S. National Library of Medicine. National Institutes of Health). Adjuvant is a substance added to a drug product formulation that affects the action of the active ingredient in a predictable way. Vehicle is an excipient or a substance, preferably without therapeutic action, used as a medium to give bulk for the administration of medicines (Stedman's Medical Spellchecker, 2006 Lippincott Williams & Wilkins). Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin. The selection of these excipients and the amounts to be used will depend on the form of application of the pharmaceutical composition.

The pharmaceutical composition used according to the present invention can be adapted to any form of administration, be it orally or parenterally, for example pulmonarily, nasally, rectally and/or intravenously. Therefore, the formulation according to the present invention may be adapted for topical or systemic application, particularly for dermal, subcutaneous, intramuscular, intra-articular, intraperitoneal, pulmonary, buccal, sublingual, nasal, percutaneous, vaginal, oral or parenteral application. The preferred form of rectal application is by means of suppositories.

Suitable preparations for oral applications are tablets, pills, chewing gums, capsules, granules, drops or syrups. Suitable preparations for parenteral applications are solutions, suspensions, reconstitutable dry preparations or sprays.

The combination of the invention may be formulated as deposits in dissolved form or in patches, for percutaneous application. Skin applications include ointments, gels, creams, lotions, suspensions or emulsions.

The combination of the invention may be formulated for its simultaneous, separate or sequential administration, with at least a pharmaceutically acceptable excipient. This has the implication that the combination of the Sigma ligand, such as a Sigma ligand of general formula (I), and the NSAID may be administered:
a) As a combination that is being part of the same medicament formulation, both being then administered always simultaneously.
b) As a combination of two units, each with one of them giving rise to the possibility of simultaneous, sequential or separate administration. In a particular embodiment, the Sigma ligand of general formula (I) is independently administered from the NSAID (i.e in two units) but at the same time. In another particular embodiment, the sigma ligand of general formula (I) is administered first, and then the NSAID is separately or sequentially administered. In yet another particular embodiment, the NSAID is administered first, and then the Sigma ligand of general formula (I) is administered, separately or sequentially, as defined.

In a particular embodiment of the present invention, the pain is selected from peripheral neuropathic pain, allodynia, causalgia, hyperalgesia, hyperesthesia, hyperpathia, neuralgia, neuritis or neuropathy. More preferably, the pain is hyperalgesia or mechanical allodynia.

“Neuropathic pain” is defined by the IASP as “pain initiated or caused by a primary lesion or dysfunction in the nervous system” (IASP, Classification of chronic pain, 2^{nd} Edition, IASP Press (2002), 210). For the purpose of this invention this term is to be treated as synonymous to “Neurogenic Pain” which is defined by the IASP as “pain initiated or caused by a primary lesion, dysfunction or transitory perturbation in the peripheral or central nervous system”.

According to the IASP “peripheral neuropathic pain” is defined as “a pain initiated or caused by a primary lesion or dysfunction in the peripheral nervous system” and “peripheral neurogenic pain” is defined as “a pain initiated or caused by a primary lesion, dysfunction or transitory perturbation in the peripheral nervous system” (IASP, Classification of chronic pain, 2^{nd} Edition, IASP Press (2002), 213).

According to the IASP “allodynia” is defined as “a pain due to a stimulus which does not normally provoke pain” (IASP, Classification of chronic pain, 2^{nd} Edition, IASP Press (2002), 210).

According to the IASP “causalgia” is defined as “a syndrome of sustained burning pain, allodynia and hyperpathia after a traumatic nerve lesion, often combined with vasomotor and sudomotor dysfunction and later trophic changes” (IASP, Classification of chronic pain, 2^{nd} Edition, IASP Press (2002), 210).

According to the IASP “hyperalgesia” is defined as “an increased response to a stimulus which is normally painful” (IASP, Classification of chronic pain, 2^{nd} Edition, IASP Press (2002), 211).

According to the IASP “hyperesthesia” is defined as “increased sensitivity to stimulation, excluding the senses” (IASP, Classification of chronic pain, 2^{nd} Edition, IASP Press (2002), 211).

According to the IASP “hyperpathia” is defined as “a painful syndrome characterized by an abnormally painful reaction to a stimulus, especially a repetitive stimulus, as well as an increased threshold” (IASP, Classification of chronic pain, 2^{nd} Edition, IASP Press (2002), 212).

The IASP draws the following difference between “allodynia”, “hyperalgesia” and “hyperpathia” (IASP, Classification of chronic pain, 2^{nd} Edition, IASP Press (2002), 212):

| | | |
|---|---|---|
| Allodynia | Lowered | Stimulus and response |
| | threshold | mode differ |
| Hyperalgesia | Increased | Stimulus and response |
| | response | rate are the same |
| Hyperpathia | Raised threshold | Stimulus and response rate |
| | Increased response | may be the same or |
| | | different |

According to the IASP “neuralgia” is defined as “pain in the distribution of a nerve or nerves” (IASP, Classification of chronic pain, 2^{nd} Edition, IASP Press (2002), 212).

According to the IASP “neuritis” is defined as “inflammation of a nerve or nerves” (IASP, Classification of chronic pain, 2^{nd} Edition, IASP Press (2002), 212).

According to the IASP “neuropathy/neuritis” is defined as “a disturbance of function or pathological change in a nerve: in one nerve mononeuropathy, in several nerves mononeuropthy multiplex, if diffuse and bilateral, polyneuropathy” (IASP, Classification of chronic pain, 2^{nd} Edition, IASP Press (2002), 212).

In a preferred embodiment of the present invention, pain refers specifically to “post-operative pain”. “Post-operative pain” refers to pain arising or resulting from an external trauma or injury such as a cut, puncture, incision, tear, or wound into tissue of an individual (including those that arise from all surgical procedures, whether invasive or non-invasive).

Another aspect of the invention is a method of treatment and/or prophylaxis of a patient suffering from pain, or likely to suffer pain, the method comprising administering to the patient in need of such a treatment or prophylaxis a therapeutically effective amount of a combination comprising at least one Sigma ligand as defined above, or a pharmaceutically acceptable salt, isomer, prodrug or solvate thereof, and at least one NSAID. By an “effective” amount or a “therapeutically effective amount” of a drug or pharmacologically active agent is meant a nontoxic but sufficient amount of the drug or agent to provide the desired effect. In the combination therapy of the present invention, an “effective amount” of one component of the combination (i.e. Sigma ligand or NSAID) is the amount of that compound that is effective to provide the desired effect when used in combination with the other component of the combination (i.e. NSAID or Sigma ligand). The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation.

According to the present invention the dosage of the NSAID can be reduced when combined with a Sigma ligand, and therefore attaining the same analgesic effect with a reduced dosage, and thus attenuating the adverse effects.

For example, the dosage regime that must be administered to the patient will depend on the patient's weight, the type of application, the condition and severity of the disease. A preferred dosage regime comprises an administration of a Sigma compound within a range of 0.5 to 100 mg/kg and of the NSAID from 0.15 to 15 mg/kg. The administration may be performed once or in several occasions.

Having described the present invention in general terms, it will be more easily understood by reference to the following examples which are presented as an illustration and are not intended to limit the present invention.

### EXAMPLES

### Example 1

### Synthesis of 4-{2-[5-Methyl-1-(naphthalen-2-yl)-1H-pyrazol-3-yloxy]ethyl}morpholine (compound 63) and its hydrochloride salt

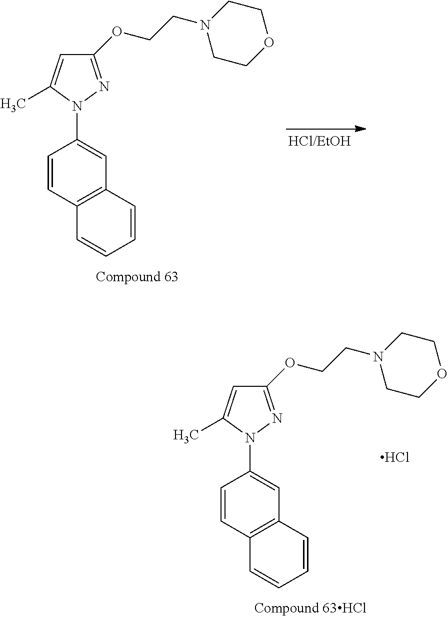 [SMILES: Cc1cc(OCCN2CCOCC2)nn1-c1ccc2ccccc2c1 ; Cc1cc(OCCN2CCOCC2)nn1-c1ccc2ccccc2c1 ; Cl]

Compound 63 can be prepared as disclosed in the previous application WO2006/021462. Its hydrochloride can be obtained according the following procedure:

Compound 63 (6.39 g) was dissolved in ethanol saturated with HCl, the mixture was stirred then for some minutes and evaporated to dryness. The residue was crystallized from isopropanol. The mother liquors from the first crystallization afforded a second crystallization by concentrating. Both crystallizations taken together yielded 5.24 g (63%) of the corresponding hydrochloride salt (m.p.=197-199° C.)

¹H-NMR (DMSO-d₆) δ ppm: 10.85 (bs, 1H), 7.95 (m, 4H), 7.7 (dd, J=2, 2, 8.8 Hz, 1H), 7.55 (m, 2H), 5.9 (s, 1H), 4.55 (m, 2H), 3.95 (m, 2H), 3.75 (m, 2H), 3.55-3.4 (m, 4H), 3.2 (m, 2H), 2.35 (s, 3H).

HPLC purity: 99.8%

### Example 2

### Assessment of Analgesia in the Treatment Post-Operative Pain

### 2.1 General Protocol.

The induction of anesthesia in rats was performed with 3% isofluran for veterinary use, employing an Ohmeda vaporizer and an anesthesia chamber. Anesthesia was kept during the surgical operation by a tube which directs the isofluran vapors to the animal's snout. Once the rats were anesthetized, they were laid down in a prone position and their right hind paws were cleaned out with alcohol.

Then, a skin incision in the hindpaw of about 10 mm was made by means of a scalpel, starting about 5 mm from the heel and extending toward the toes. Fascia was located and by means of curve scissors muscle was elevated and a longitudinal incision of about 5 mm was made, thus the muscle origin and insertion remained intact. The skin of the paw was stitched with a suturing stitch with breaded silk (3.0) and the wound was cleaned out with povidone.

The assessment was performed 30 minutes after the administration of product and always 4 hours after the plantar incision. The analysis was carried out evaluating the mechanical allodynia. It was tested using von Frey filaments: Animals were placed in methacrylate cylinders on an elevated surface, with metallic mesh floor perforated in order to apply the filaments. After an acclimation period of about 30 minutes within the cylinders, both hindpaws were stimulated (the injured and the non-injured paw, serving the latter as control), starting with the lowest force filament (0.4 g) and reaching a 15 g filament. The animal's response to pain was manifested by the withdrawal of the paw as a consequence of the painful stimulus caused by a filament.

### 2.1 Sigma Antagonists: BD1063 and Compound 63.HCl

The efficacy of selective Sigma-1 receptor antagonists BD1063 (1-[2-(3,4-dichlorophenyl)ethyl]-4-methylpiperazine) supplied by Tocris Cookson Ltd. (Bristol, UK) and compound 63.HCL in rats was evaluated separately as follows:

1) BD1063 was administered at different doses (20, 40 and 80 mg/kg) and
2) compound 63.HCl was administered at different doses (10, 20, 40 and 80 mg/kg). Both administrations were performed 3.5 hours after surgery.

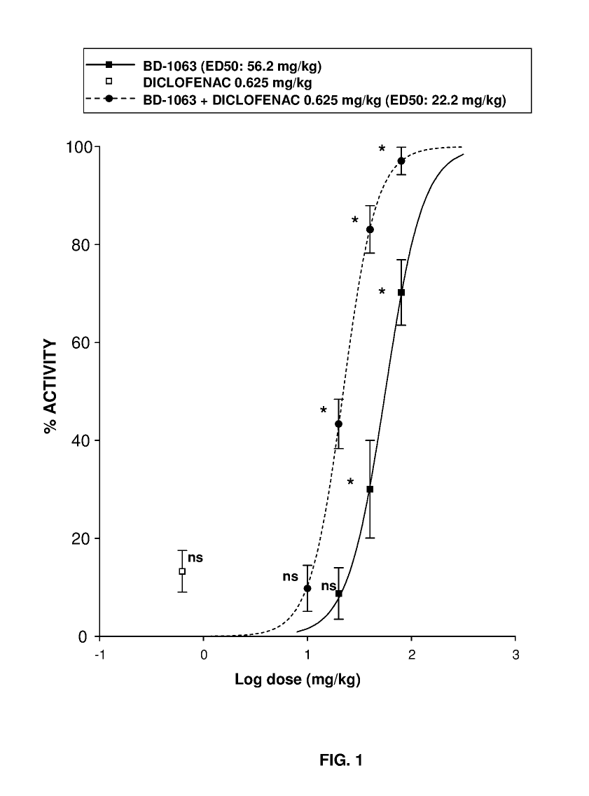

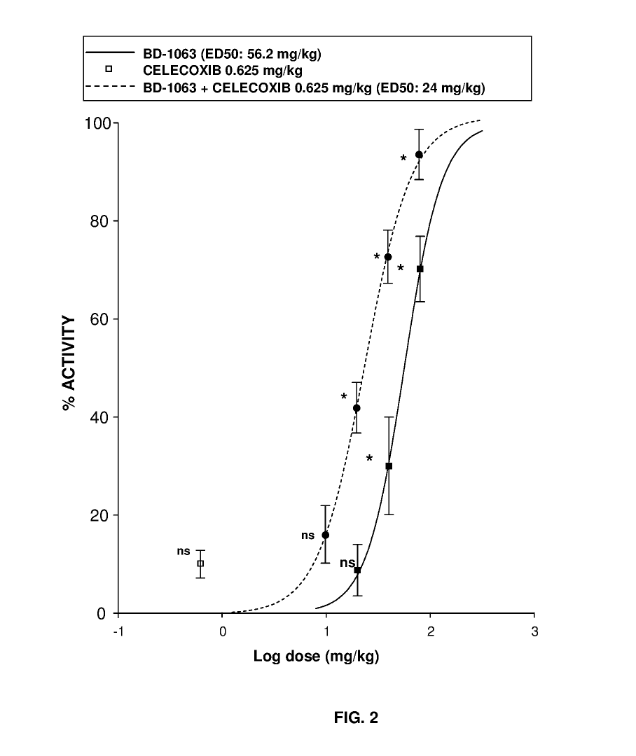

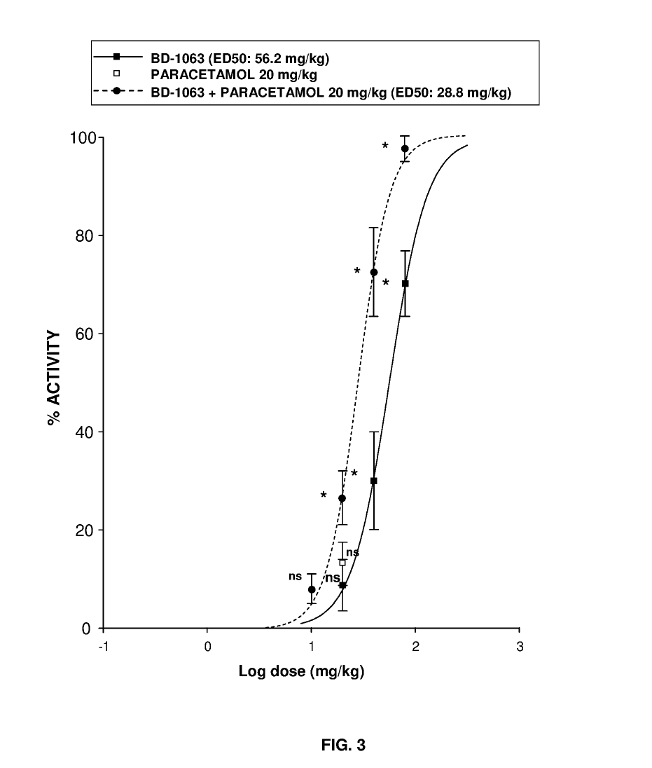

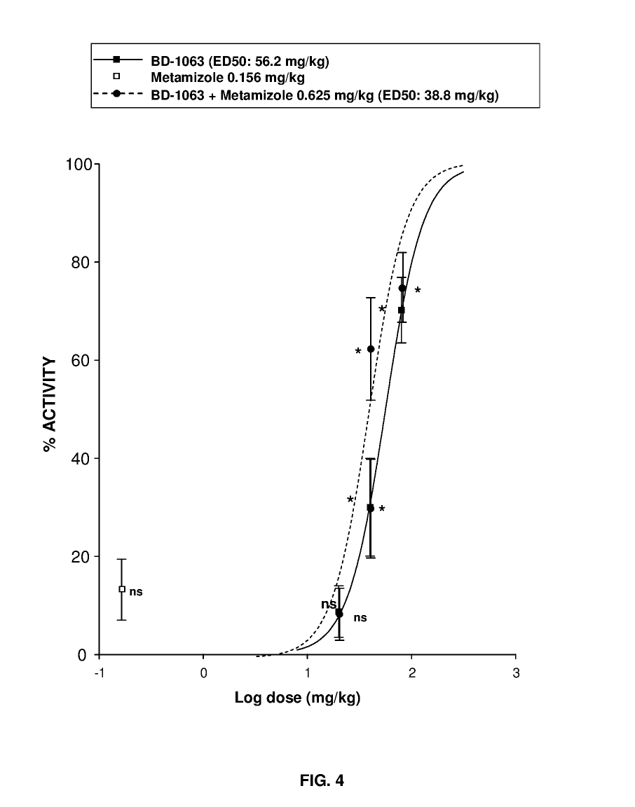

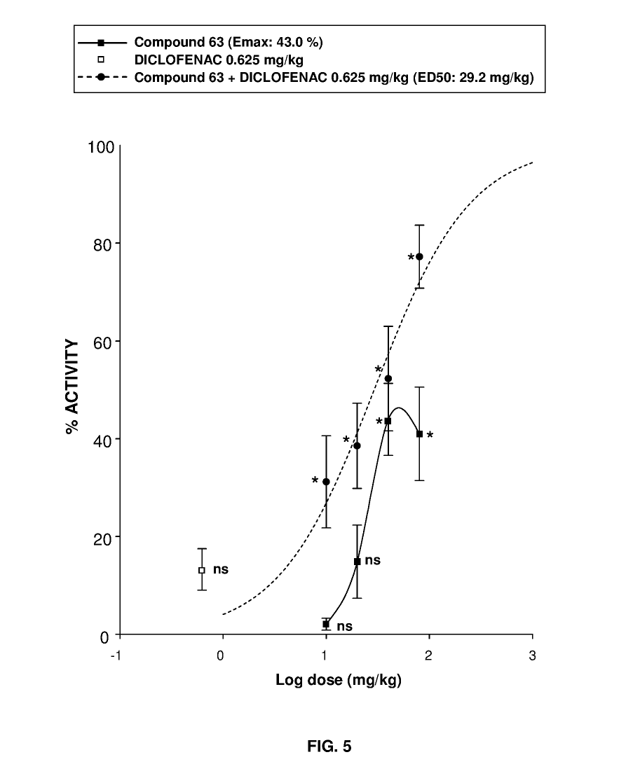

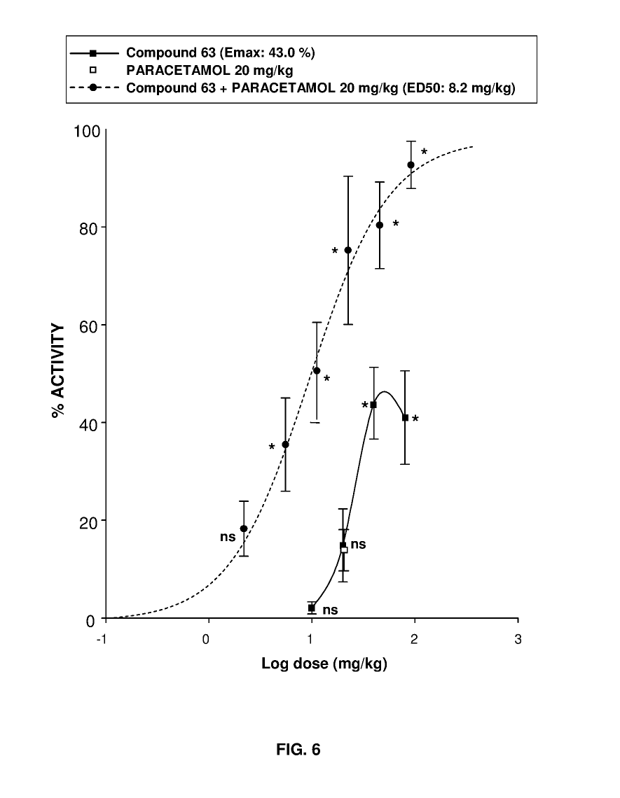

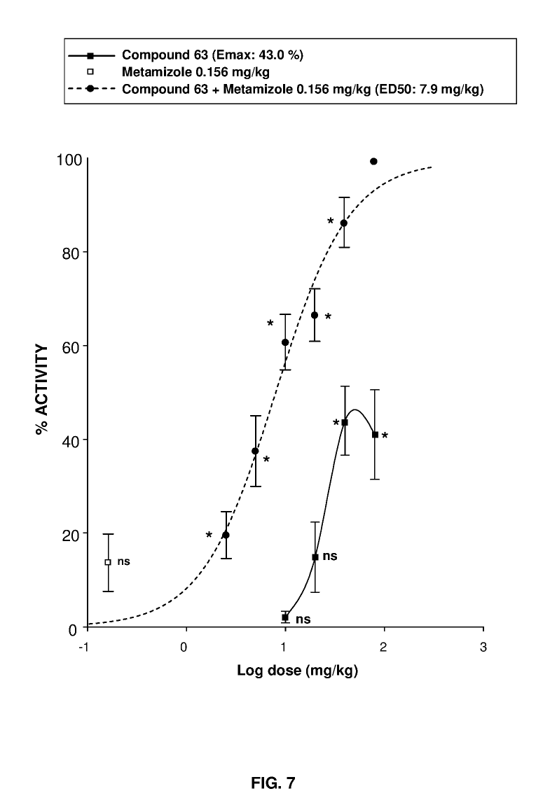

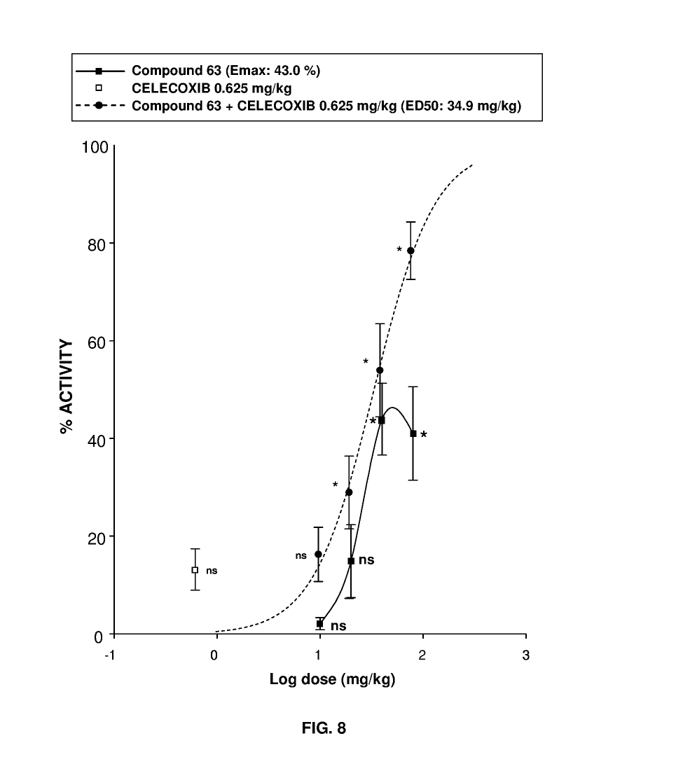

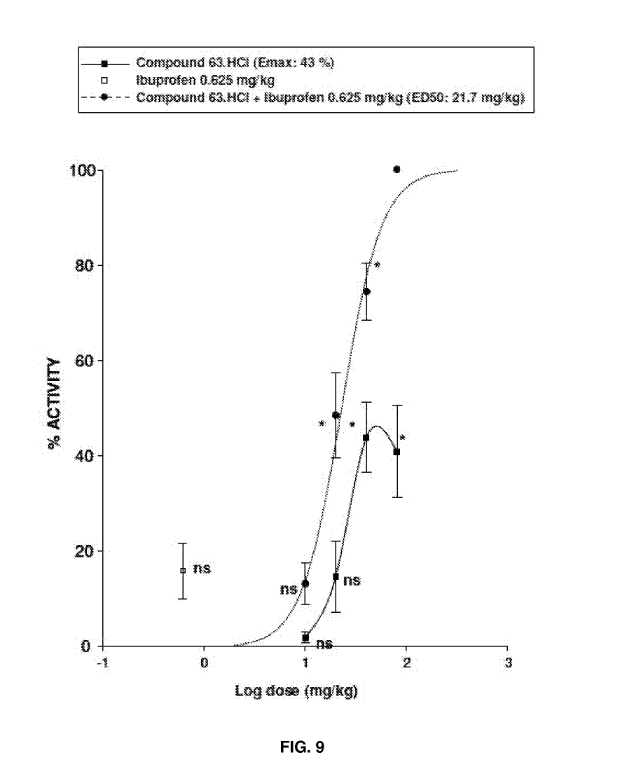

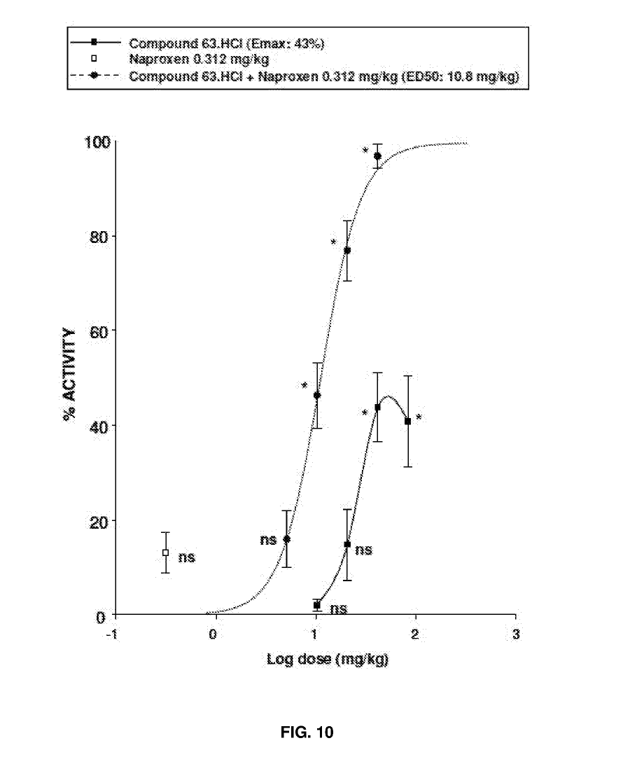

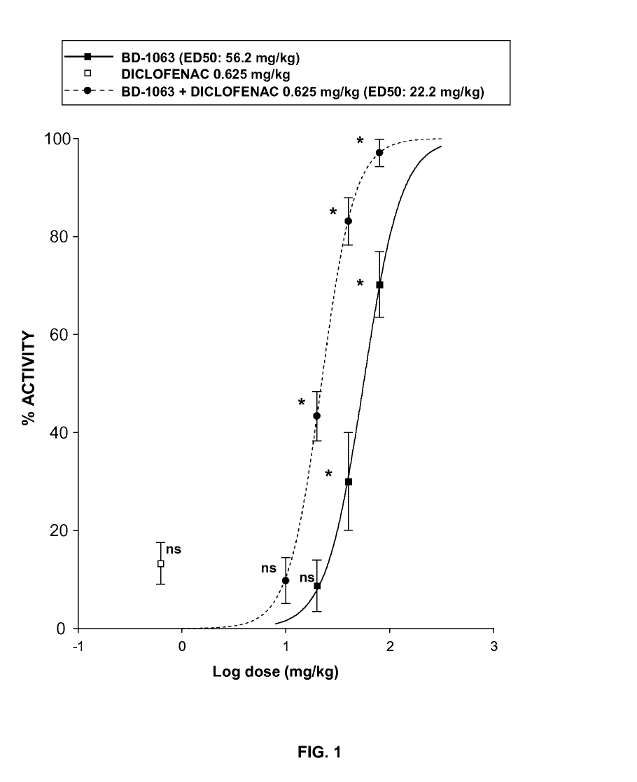

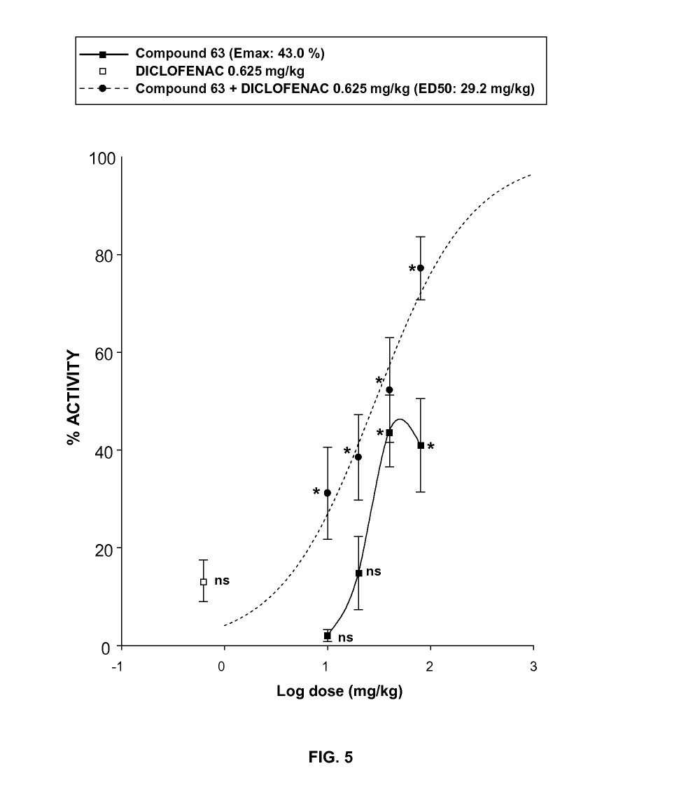

The treated subjects were tested according to the mechanical allodynia protocol above. BD1063 produced a dose-dependent effect with ED50=56.2 mg/kg (FIGS. 1, 2, 3 and 4) and compound 63.HCl produced a dose dependent effect with a maximum effect of 43% (FIGS. 5, 6, 7, 8, 9 and 10).

### 2.2 NSAID: Diclofenac (FIGS. 1 and 5)

The efficacy of Diclofenac, BD1063 and compound 63.HCl was evaluated separately as follows:
Diclofenac was administered at a constant dose of 0.625 mg/kg;
BD1063 alone was administered at different doses (20, 40 and 80 mg/kg); and
compound 63.HCl alone was administered at different doses (10, 20, 40 and 80 mg/kg).

Subsequently, the efficacy of the combined use of Diclofenac and BD1063 was assayed at different doses of BD1063 (10, 20, 40 and 80 mg/kg), while the Diclofenac dose remained constant (0.625 mg/kg) (FIG. 1). The efficacy of the combined use of Diclofenac and compound 63.HCl was assayed at different doses of compound 63.HCl (10, 20, 40 and 80 mg/kg), while the Diclofenac dose remained constant (0.625 mg/kg) (FIG. 5).

The administrations were performed simultaneously 3.5 hours after surgery. The treated subjects were tested according to the mechanical allodynia protocol above. Diclofenac (0.312 mg/kg) alone produced no significant effect (ns). BD1063 produced significant effect only at 40 and 80 mg/kg. Compound 63.HCl produced significant effect only at 40 and 80 mg/kg.

As to the combinations, the combination Diclofenac+BD1063 produced a dose-dependent effect with ED50=22.2 mg/kg; and the combination Diclofenac+compound 63.HCl produced a dose-dependent effect with ED50=29.2 mg/kg. Therefore, BD1063 and compound 63.HCl enhance Diclofenac analgesia in the treatment of post-operative pain. Significantly, combinations of a sub-active dose of Diclofenac (0.625 mg/kg) and compound 63.HCl (10, 20, 40 and 80 mg/kg) administered 3.5 hours after surgery, result in an increase in the analgesic activity which is greater than the sum of the activities of each component, both related to potency (shift to the left of the dose-response curve for compound 63.HCl) and efficacy (reaching 77%, whereas maximum efficacy without the sub-active dose of Diclofenac is 43%).

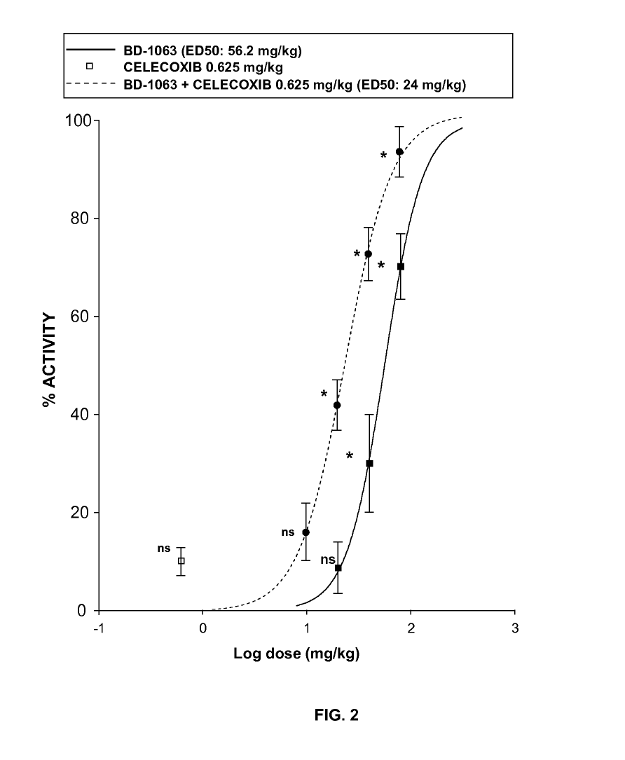

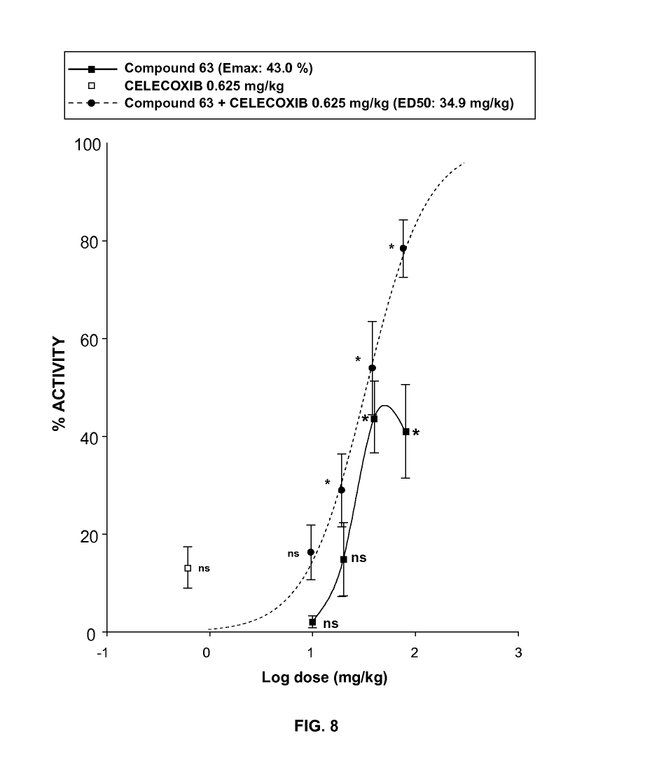

### 2.3 NSAID: Celecoxib (FIGS. 2 and 8)

The efficacy of Celecoxib, BD1063 and compound 63.HCl was evaluated separately as follows:
Celecoxib was administered at a constant dose of 0.625 mg/kg;
BD1063 alone was administered at different doses (20, 40 and 80 mg/kg); and
compound 63.HCl alone was administered at different doses (10, 20, 40 and 80 mg/kg).

Subsequently, the efficacy of the combined use of Celecoxib and BD1063 was assayed at different doses of BD1063 (10, 20, 40 and 80 mg/kg), while the Celecoxib dose remained constant (0.625 mg/kg) (FIG. 2). The efficacy of the combined use of Celecoxib and compound 63.HCl was assayed at different doses of compound 63.HCl (10, 20, 40 and 80 mg/kg), while the Celecoxib dose remained constant (0.625 mg/kg) (FIG. 8).

The administrations were performed simultaneously 3.5 hours after surgery. The treated subjects were tested according to the mechanical allodynia protocol above.

Celecoxib (0.625 mg/kg) alone produced no significant effect (ns). BD1063 produced significant effect only at 40 and 80 mg/kg. Compound 63.HCl produced significant effect only at 40 and 80 mg/kg.

As to the combinations, the combination Celecoxib+BD1063 produced a dose-dependent effect with ED50=24 mg/kg; and the combination Celecoxib+compound 63.HCl produced a dose-dependent effect with ED50=34.9 mg/kg. Therefore, BD1063 and compound 63.HCl enhance Celecoxib analgesia in the treatment of post-operative pain. Significantly, combinations of a sub-active dose of Celecoxib (0.625 mg/kg) and compound 63.HCl (10, 20, 40 and 80 mg/kg) administered 3.5 hours after surgery, result in an increase in the analgesic activity which is greater than the sum of the activities of each component, both related to potency (shift to the left of the dose-response curve for compound 63.HCl) and efficacy (reaching 79%, whereas maximum efficacy without the sub-active dose of Celecoxib is 43%).

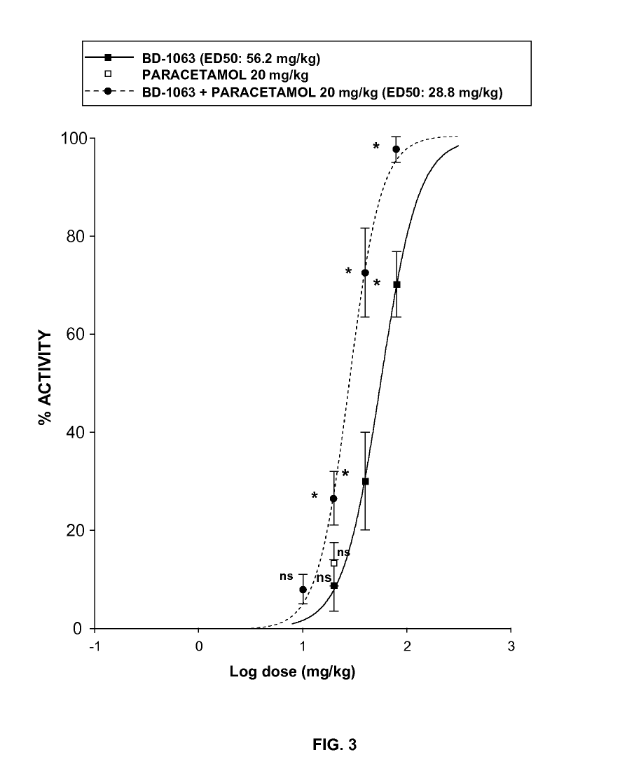

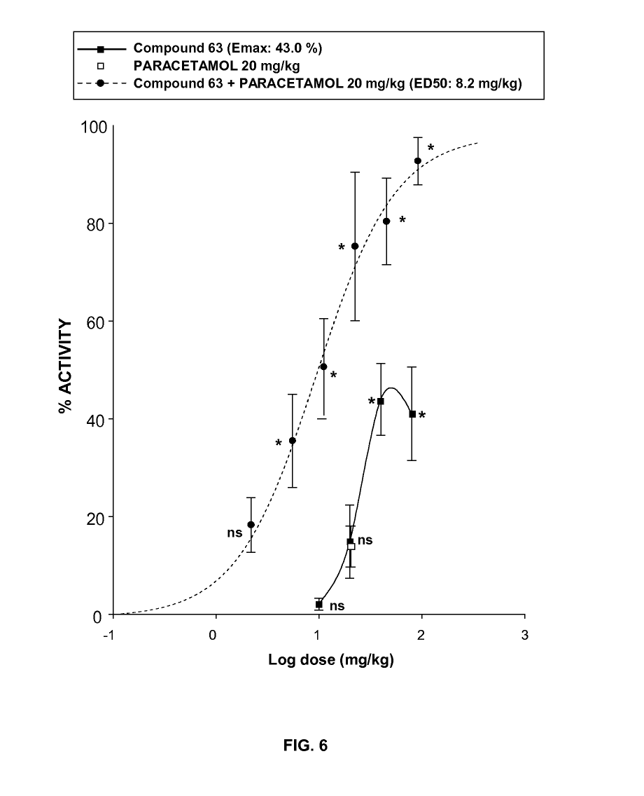

### 2.4 NSAID: Paracetamol (FIGS. 3 and 6)

The efficacy of Paracetamol, BD1063 and compound 63.HCl was evaluated separately as follows:
Paracetamol was administered at a constant dose of 20 mg/kg;
BD1063 alone was administered at different doses (20, 40 and 80 mg/kg); and
compound 63.HCl alone was administered at different doses (10, 20, 40 and 80 mg/kg).

Subsequently, the efficacy of the combined use of Paracetamol and BD1063 was assayed at different doses of BD1063 (10, 20, 40 and 80 mg/kg), while the Paracetamol dose remained constant (20 mg/kg) (FIG. 3). The efficacy of the combined use of Paracetamol and compound 63.HCl was assayed at different doses of compound 63.HCl (5, 10, 20, 40 and 80 mg/kg), while the Paracetamol dose remained constant (20 mg/kg) (FIG. 6).

The administrations were performed simultaneously 3.5 hours after surgery. The treated subjects were tested according to the mechanical allodynia protocol above.

Paracetamol (20 mg/kg) alone produced no significant effect (ns). BD1063 produced significant effect only at 40 and 80 mg/kg. Compound 63.HCl produced significant effect only at 40 and 80 mg/kg.

As to the combinations, the combination Paracetamol+BD1063 produced a dose-dependent effect with ED50=28.8 mg/kg; and the combination Paracetamol+compound 63.HCl produced a dose-dependent effect with ED50=8.2 mg/kg. Therefore, BD1063 and compound 63.HCl enhance Paracetamol analgesia in the treatment of post-operative pain. Significantly, combinations of a sub-active dose of Paracetamol (20 mg/kg) and compound 63.HCl (5, 10, 20, 40 and 80 mg/kg) administered 3.5 hours after surgery, result in an increase in the analgesic activity which is greater than the sum of the activities of each component, both related to potency (shift to the left of the dose-response curve for compound 63.HCl) and efficacy (reaching 94%, whereas maximum efficacy without the sub-active dose of Paracetamol is 43%).

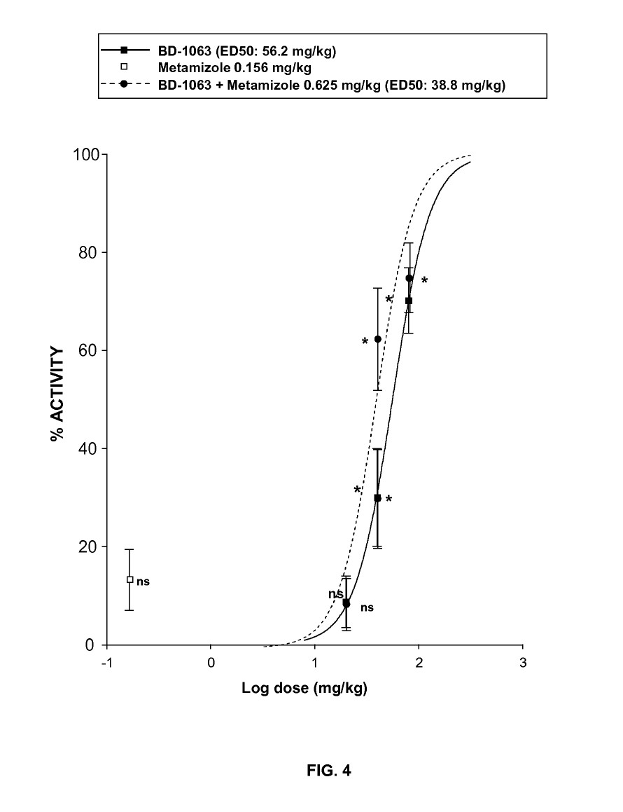

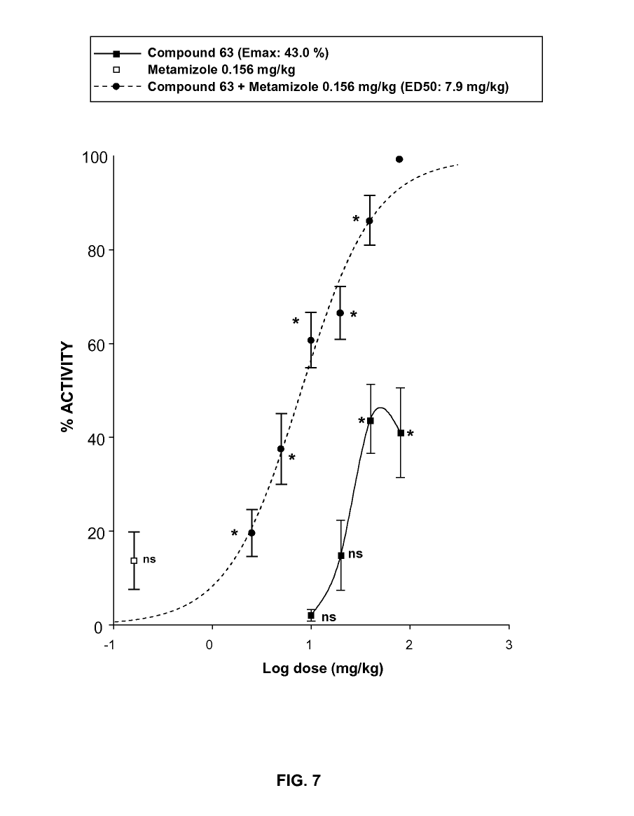

### 2.5 NSAID: Metamizole (FIGS. 4 and 7)

The efficacy of Metamizole, BD1063 and compound 63.HCl was evaluated separately as follows:
Metamizole was administered at a constant dose of 0.156 mg/kg;
BD1063 alone was administered at different doses (20, 40 and 80 mg/kg); and
compound 63.HCl alone was administered at different doses (10, 20, 40 and 80 mg/kg).

Subsequently, the efficacy of the combined use of Metamizole and BD1063 was assayed at different doses of BD1063 (10, 20, 40 and 80 mg/kg), while the Metamizole dose remained constant (0.156 mg/kg) (FIG. 4). The efficacy of the combined use of Metamizole and compound 63.HCl was assayed at different doses of compound 63.HCl (5, 10, 20, 40 and 80 mg/kg), while the Metamizole dose remained constant (0.156 mg/kg) (FIG. 7).

The administrations were performed simultaneously 3.5 hours after surgery. The treated subjects were tested according to the mechanical allodynia protocol above.

Metamizole (0.156 mg/kg) alone produced no significant effect (ns). BD1063 produced significant effect only at 40 and 80 mg/kg. Compound 63.HCl produced significant effect only at 40 and 80 mg/kg.

As to the combinations, the combination Metamizole+BD1063 produced a dose-dependent effect with ED50=38.8 mg/kg; and the combination Metamizole+compound 63.HCl produced a dose-dependent effect with ED50=7.9 mg/kg. Therefore, BD1063 and compound 63.HCl enhance Metamizole analgesia in the treatment of post-operative pain. Significantly, combinations of a sub-active dose of Metamizole (0.156 mg/kg) and compound 63.HCl (5, 10, 20, 40 and 80 mg/kg) administered 3.5 hours after surgery, result in an increase in the analgesic activity which is greater than the sum of the activities of each component, both related to potency (shift to the left of the dose-response curve for compound 63.HCl) and efficacy (reaching 100%, whereas maximum efficacy without the sub-active dose of Metamizole is 43%).

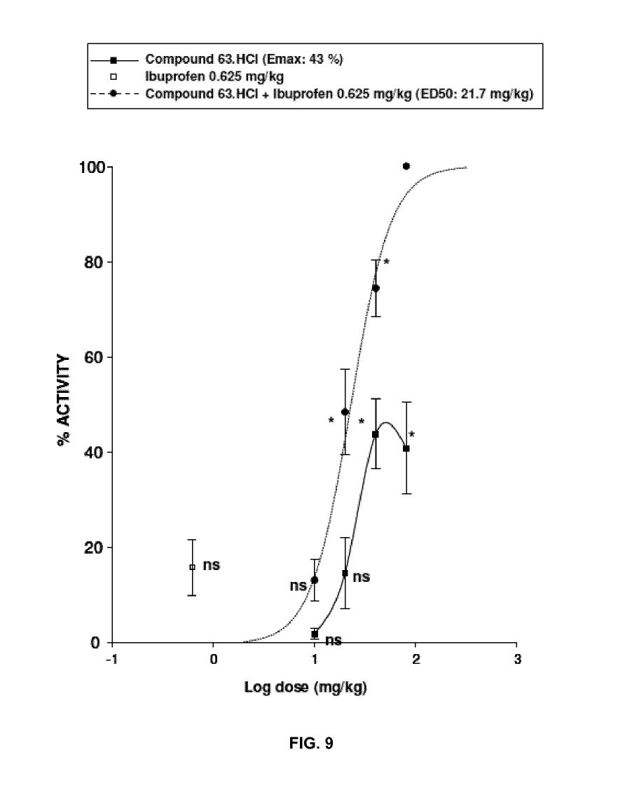

### 2.6 NSAID: Ibuprofen (FIG. 9)

The efficacy of Ibuprofen and compound 63.HCl was evaluated separately as follows:
Ibuprofen was administered at a constant dose of 0.625 mg/kg; and
compound 63.HCl alone was administered at different doses (10, 20, 40 and 80 mg/kg).

Subsequently, the efficacy of the combined use of ibuprofen and compound 63.HCl was assayed at different doses of compound 63.HCl (10, 20, 40 and 80 mg/kg), while the Ibuprofen dose remained constant (0.625 mg/kg) (FIG. 9).

The administrations were performed simultaneously 3.5 hours after surgery. The treated subjects were tested according to the mechanical allodynia protocol above.

Ibuprofen (0.625 mg/kg) alone produced no significant effect (ns). Compound 63.HCl produced significant effect only at 40 and 80 mg/kg.

As to the combinations, the combination Ibuprofen+compound 63.HCl produced a dose-dependent effect with ED50=21.7 mg/kg. Therefore, compound 63.HCl enhances Ibuprofen analgesia in the treatment of post-operative pain. Significantly, combinations of a sub-active dose of Ibuprofen (0.625 mg/kg) and compound 63.HCl (10, 20, 40 and 80 mg/kg) administered 3.5 hours after surgery, result in an increase in the analgesic activity which is greater than the sum of the activities of each component, both related to potency (shift to the left of the dose-response curve for compound 63.HCl) and efficacy (reaching 100%, whereas maximum efficacy without the sub-active dose of Ibuprofen is 43%).

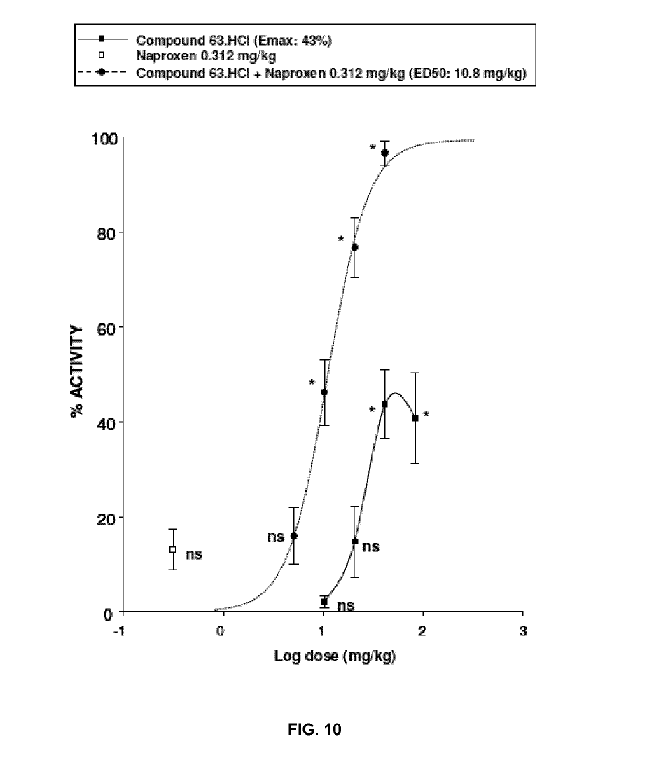

### 2.7 NSAID: Naproxen (FIG. 10)

The efficacy of Naproxen and compound 63.HCl was evaluated separately as follows:
Naproxen was administered at a constant dose of 0.312 mg/kg; and
compound 63.HCl alone was administered at different doses (10, 20, 40 and 80 mg/kg).

Subsequently, the efficacy of the combined use of Naproxen and compound 63.HCl was assayed at different doses of compound 63.HCl (5, 10, 20 and 40 mg/kg), while the Naproxen dose remained constant (0.312 mg/kg) (FIG. 10).

The administrations were performed simultaneously 3.5 hours after surgery. The treated subjects were tested according to the mechanical allodynia protocol above.

Naproxen (0.312 mg/kg) alone produced no significant effect (ns). Compound 63.HCl produced significant effect only at 40 and 80 mg/kg.

As to the combinations, the combination Naproxen+compound 63.HCl produced a dose-dependent effect with ED50=10.8 mg/kg. Therefore, compound 63.HCl enhances Naproxen analgesia in the treatment of post-operative pain. Significantly, combinations of a sub-active dose of Naproxen (0.312 mg/kg) and compound 63.HCl (5, 10, 20 and 40 mg/kg) administered 3.5 hours after surgery, result in an increase in the analgesic activity which is greater than the sum of the activities of each component, both related to potency (shift to the left of the dose-response curve for compound 63.HCl) and efficacy (reaching 97%, whereas maximum efficacy without the sub-active dose of Naproxen is 43%)

The following table summarizes all the results:

| | Mean of the porcentage of analgesia in the post-operative pain model (%) | | | | | | |
|---|---|---|---|---|---|---|---|
| Dose | Compounds alone | | | | | | |
| (mg/kg) | compound 63 | naproxen | ibuprofen | metamizole | celecoxib | diclofenac | paracetamol |
| 80 | 41 | | | | | | |
| 40 | 43 | | | | | | |
| 20 | 15 | | | | | | 13 |
| 10 | 2 | | | | | | |
| 5 | | | | | | | |
| 2.5 | | | | | | | |
| 1.25 | | | | | | | |
| 0.625 | | | 16 | | 13 | 13 | |
| 0.312 | | 13 | | | | | |
| 0.156 | | | | 14 | | | |

| | Mean of the porcentage of analgesia in the post-operative pain model (%) | | | | | |
|---|---|---|---|---|---|---|
| Dose | Combination of compound 63 + subactive dose of NSAIDs | | | | | |
| (mg/kg) | +naproxen | +ibuprofen | +metamizole | +celecoxib | +diclofenac | +paracetamol |
| 80 | | 100 | 100 | 79 | 77 | 94 |
| 40 | 97 | 74 | 87 | 54 | 52 | 82 |
| 20 | 77 | 49 | 67 | 29 | 39 | 77 |
| 10 | 46 | 13 | 61 | 16 | 31 | 52 |
| 5 | 16 | | 38 | | | 37 |
| 2.5 | | | 20 | | | 19 |
| 1.25 | | | | | | |
| 0.625 | | | | | | |
| 0.312 | | | | | | |
| 0.156 | | | | | | |

### REFERENCES

Botting-R M; Clinical Infectious Diseases; 2000; 31:S202-10
Cobos, E. J. et al.; Curr. Neuropharmacol.; 2008; 6, 344-366.
Dugowson et al.; Phys. Med. Rehabil. Clin. N. Am.; 2006 17 347-354
Hellewell, S. B. and Bowen, W. D.; Brain Res.; 1990; 527, 244-253
Herndon et al.; Pharmacotherapy; 2008; 28(6), 788-805
Hinz et al.; FASEB Journal; 2007; 7, 2343-2351
Leitner, M. L.; Eur. J. Pharmacol.; 1994; 259; 65-69
Maurice, T. et al.; Pharmacol. Ther.; 2009; 124, 195-206
Prasad, P. D. et al.; J. Neurochem.; 1998; 70 (2); 443-451
Quiron, R. et al.; Trends Pharmacol. Sci.; 1992; 13:85-86
Romero, L., et al.; Br. J. Pharmacol.; 2012; doi: 10.1111/j.1476-5381
Ronsisvalle, G. et al.; Pure Appl. Chem.; 2001; 73; 1499-1509
Telleria-Diaz et al.; Pain; 2010 148, 26-35

1-16. (canceled)

17. A synergistic combination comprising at least one Sigma ligand and at least one Non-steroidal Anti-inflammatory Drug (NSAID).

18. The synergistic combination according to claim 17, wherein the at least one sigma ligand has a general formula (I) 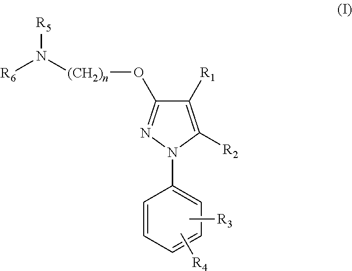
wherein,
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)—R₈, —NR₈R₉, —NR₈C(O)R₉, —NO₂, —N═CR₈R₉, and halogen, or together with the phenyl they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₈, and halogen:
or together with the nitrogen atom to which they are attached, form a substituted or unsubstituted, aromatic or non-aromatic heterocyclyl group;
n is selected from 1, 2, 3, 4, 5, 6, 7 and 8;
t is 0, 1 or 2;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen,
or a pharmaceutically acceptable salt, isomer, prodrug or solvate thereof.

19. The synergistic combination according to claim 18, wherein R₁ is selected from H, —COR₈, and substituted or unsubstituted alkyl.

20. The synergistic combination according to claim 18, wherein R₂ is H or substituted or unsubstituted alkyl.

21. The synergistic combination according to claim 18, wherein R₃ and R₄ together with the phenyl group form a naphthyl ring system.

22. The synergistic combination according to claim 18, wherein n is selected from 2, 3, and 4.

23. The synergistic combination according to claim 18, wherein R₅ and R₆, together with the nitrogen atom to which they are attached, form a morpholine-4-yl group.

24. The synergistic combination claim 18, wherein the sigma ligand of general formula (I) is selected from:
4-{2-(1-(3,4-dichlorophenyl)-5-methyl-1H pyrazol-3-yloxy)ethyl}morpholine,
2-[1-(3,4-Dichlorophenyl)-5-methyl-1H-pyrazol-3-yloxy]-N,N-diethylethanamine,
1-(3,4-Dichlorophenyl)-5-methyl-3-[2-(pyrrolidin-1-yl)ethoxy]-1H-pyrazole,
1-(3,4-Dichlorophenyl)-5-methyl-3-[3-(pyrrolidin-1-yl)propoxy]-1H-pyrazole,
1-{2-[1-(3,4-Dichlorophenyl)-5-methyl-1H-pyrazol-3-yloxy]ethyl}piperidine,
1-{2-[1-(3,4-dichlorophenyl)-5-methyl-1H-pyrazol-3-yloxy]ethyl}-1H-imidazole,
3-{1-[2-(1-(3,4-Dichlorophenyl)-5-methyl-1H-pyrazol-3-yloxy)ethyl]piperidin-4-yl}-3H-imidazo[4,5-b]pyridine,
1-{2-[1-(3,4-Dichlorophenyl)-5-methyl-1H-pyrazol-3-yloxy]ethyl}-4-methylpiperazine,
Ethyl 4-{2-[1-(3,4-dichlorophenyl)-5-methyl-1H-pyrazol-3-yloxy]ethyl}piperazine carboxylate,
[1-(4-(2-(1-(3,4-dichlorophenyl)-5-methyl-1H-pyrazol-3-yloxy)ethyl)piperazin-1-yl)ethanone,
4-{2-(1-(4-Methoxyphenyl)-5-methyl-1H-pyrazol-3-yloxy]ethyl}morpholine,
1-(4-Methoxyphenyl)-5-methyl-3-[2-(pyrrolidin-1-yl)ethoxy]-1H-pyrazole,
1-(4-Methoxyphenyl)-5-methyl-3-[3-(pyrrolidin-1-yl)propoxy]-1H-pyrazole,
1-[2-(1-(4-Methoxyphenyl)-5-methyl-1H-pyrazol-3-yloxy)ethyl]piperidine,
1-{2-[1-(4-Methoxyphenyl)-5-methyl-1H-pyrazol-3-yloxy]ethyl}-1H-imidazole,
4-{2-[1-(3,4-Dichlorophenyl)-5-phenyl-1H-pyrazol-3-yloxy]ethyl}morpholine,
1-(3,4-Dichlorophenyl)-5-phenyl-3-[2-(pyrrolidin-1-yl)ethoxy]-1H-pyrazole,
[1-(3,4-Dichlorophenyl)-5-phenyl-3-[3-(pyrrolidin-1-yl)propoxy]-1H-pyrazole,
1-{2-[1-(3,4-Dichlorophenyl)-5-phenyl-1H-pyrazol-3-yloxy]ethyl}piperidine,
1-{2-(1-(3,4-Dichlorophenyl)-5-phenyl-1H-pyrazol-3-yloxy]ethyl}-1H-imidazole,
2-{2-[1-(3,4-dichlorophenyl)-5-phenyl-1H-pyrazol-3-yloxy]ethyl}-1,2,3,4-tetrahydroisoquinoline,
4-{4-[1-(3,4-Dichlorophenyl)-5-methyl-1H-pyrazol-3-yloxy]butyl}morpholine,
1-(3,4-Dichlorophenyl)-5-methyl-3-[4-(pyrrolidin-1-yl)butoxy]-1H-pyrazole,
1-{4-[1-(3,4-Dichlorophenyl)-5-methyl-1H-pyrazol-3-yloxy]butyl}piperidine,
1-{4-[1-(3,4-Dichlorophenyl)-5-methyl-1H-pyrazol-3-yloxy]butyl}-4-methylpiperazine,
1-{4-[1-(3,4-Dichlorophenyl)-5-methyl-1H-pyrazol-3-yloxy]butyl}-1H-imidazole,
4-[1-(3,4-Dichlorophenyl)-5-methyl-1H-pyrazol-3-yloxy]-N, N-diethylbutan-1-amine,
1-{4-[1-(3,4-dichlorophenyl)-5-methyl-1H-pyrazol-3-yloxy]butyl}-4-phenylpiperidine,
1-{4-[1-(3,4-dichlorophenyl)-5-methyl-1H-pyrazol-3-yloxy]butyl}-6,7-dihydro-1H-indol-4(5H)-one,
2-{4-[1-(3,4-dichlorophenyl)-5-methyl-1H-pyrazol-3-yloxy]butyl}-1,2,3,4-tetrahydroisoquinoline,
4-{2-[1-(3,4-dichlorophenyl)-5-isopropyl-1H-pyrazol-3-yloxy]ethyl}morpholine,
2-[1-(3,4-Dichlorophenyl)-5-isopropyl-1H-pyrazol-3-yloxy]-N,N-diethylethanamine,
1-(3,4-Dichlorophenyl)-5-isopropyl-3-[2-(pyrrolidin-1-yl)ethoxy]-1H-pyrazole,
1-(3,4-Dichlorophenyl)-5-isopropyl-3-[3-(pyrrolidin-1-yl)propoxy]-1H-pyrazole,
1-{2-[1-(3,4-Dichlorophenyl)-5-isopropyl-1H-pyrazol-3-yloxy]ethyl}piperidine,
2-{2-[1-(3,4-dichlorophenyl)-5-isopropyl-1H-pyrazol-3-yloxy]ethyl}-1,2,3,4-tetrahydroisoquinoline,
4-{2-[1-(3,4-dichlorophenyl)-1H-pyrazol-3-yloxy]ethyl}morpholine,
2-[1-(3,4-dichlorophenyl)-1H-pyrazol-3-yloxy]N,N-diethylethanamine,
1-(3,4-dichlorophenyl)-3-[2-(pyrrolidin-1-yl)ethoxy]-1H-pyrazole,
1-{2-[1-(3,4-dichlorophenyl)-1H-pyrazol-3-yloxy]ethyl}piperidine,
1-(3,4-dichlorophenyl)-3-[3-(pyrroidin-1-yl)propoxy]-1H-pyrazole,
1-{2-[1-(3,4-Dichlorophenyl)-5-methyl-1H-pyrazol-3-yloxy]ethyl}piperazine,
1-{2-[1-(3,4-Dichlorophenyl)-5-methyl-1H-pyrazol-3-yloxy]ethyl}pyrrolidin-3-amine,
4-{2-[1-(3,4-Dichlorophenyl)-4,5-dimethyl-1H-pyrazol-3-yloxy]ethyl}morpholine,
2-[1-(3,4-Dichlorophenyl)-4,5-dimethyl-1H-pyrazol-3-yloxy]-N,N-diethylethanamine,
1-(3,4-Dichlorophenyl)-4,5-dimethyl-3-[2-(pyrrolidin-1-yl)ethoxy]-1H-pyrazole,
1-(3,4-Dichlorophenyl)-4,5-dimethyl-3-[3-(pyrrolidin-1-yl)propoxy]-1H-pyrazole,
1-{2-[1-(3,4-Dichlorophenyl)-4,5-dimethyl-1H-pyrazol-3-yloxy]ethyl}piperidine,
4-{4-[1-(3,4-dichlorophenyl)-1H-pyrazol-3-yloxy]butyl}morpholine,
(2S,6R)-4-{4-[1-(3,4-dichlorophenyl)-1H-pyrazol-3-yloxy]butyl}-2,6-dimethylmorpholine,
1-{4-[1-(3,4-Dichlorophenyl)-1H-pyrazol-3-yloxy]butyl}piperidine,
1-(3,4-Dichlorophenyl)-3-[4-(pyrroidin-1-yl)butoxy]-1H-pyrazole,
4-[1-(3,4-dichlorophenyl)-1H-pyrazol-3-yloxy]-N,N-diethylbutan-1-amine,
N-benzyl-4-[1-(3,4-dichlorophenyl)-1H-pyrazol-3-yloxy]-N-methylbutan-1-amine,
4-[1-(3,4-dichlorophenyl)-1H-pyrazol-3-yloxy]-N-(2-methoxyethyl)-N-methylbutan-1-amine,
4-{4-[1-(3,4-dichlorophenyl)-1H-pyrazol-3-yloxy]butyl}thiomorpholine,
1-[1-(3,4-Dichlorophenyl)-5-methyl-3-(2-morpholinoethoxy)-1H-pyrazol-4-yl]ethanone,
1-{1-(3,4-dichlorophenyl)-5-methyl-3-[2-(pyrrolidin-1-yl)ethoxy]-1H-pyrazol-4-yl}ethanone,
1-{1-(3,4-dichlorophenyl)-5-methyl-3-[2-(piperidin-1-yl)ethoxy]-1H-pyrazol-4-yl}ethanone,
1-{1-(3,4-dichlorophenyl)-3-[2-(diethylamino)ethoxy]-5-methyl-1H-pyrazol-4-yl}ethanone,
4-{2-[5-Methyl-1-(naphthalen-2-yl)-1H-pyrazol-3-yloxy]ethyl}morpholine,
N,N-Diethyl-2-[5-methyl-1-(naphthalen-2-yl)-1H-pyrazol-3-yloxy]ethanamine,
1-{2-[5-Methyl-1-(naphthalen-2-yl)-1H-pyrazol-3-yloxy]ethyl}piperidine, and
5-Methyl-1-(naphthalen-2-yl)-3-[2-(pyrrolidin-1-yl)ethoxy]-1H-pyrazole,
or a pharmaceutically acceptable salt, solvate or prodrug thereof.

25. The synergistic combination according to claim 17, wherein the NSAID is selected from the group consisting of paracetamol, ibuprofen, naproxen, ketoprofen, dexketoprofen, mefenamic acid, piroxicam, meloxicam, flurbiprofen, aceclofenac, acemetacin, aldofenac, amfenac, bendazac, bromfenac, bumadizone, bufexamac, diclofenac, difenpiramide, etodolac, felbinac, fentiazac, indomethacin, ketorolac, lonazolac, oxametacin, proglumetacin, sulindac, tolmetin, zomepirac, celecoxib, cimicoxib, deracoxib, etoricoxib, firocoxib, lumiracoxib, mavacoxib, parecoxib, robenacoxib, rofecoxib, valdecoxib, ampyrone, azapropazone, clofezone, kebuzone, metamizole, mofebutazone, nifenazone, oxyphenbutazone, phenazone, phenylbutazone, sulfinpyrazone, suxibuzone and feprazone.

26. The synergistic combination according to claim 17, wherein the NSAID is selected from the group consisting of paracetamol, ibuprofen, naproxen, diclofenac, celecoxib and metamizole.

27. The synergistic combination according to claim 17, wherein the combination comprises:
4-{2-[5-methyl-1-(naphthalen-2-yl)-1H-pyrazol-3-yloxy]ethyl}morpholine or a salt thereof and diclofenac or celecoxib; or
4-{2-[5-methyl-1-(naphthalen-2-yl)-1H-pyrazol-3-yloxy]ethyl}morpholine or a salt thereof and metamizole; or
4-{2-[5-methyl-1-(naphthalen-2-yl)-1H-pyrazol-3-yloxy]ethyl}morpholine or a salt thereof and ibuprofen or naproxen; or
4-{2-[5-methyl-1-(naphthalen-2-yl)-1H-pyrazol-3-yloxy]ethyl}morpholine or a salt thereof and paracetamol.

28. The synergistic combination according to claim 17, wherein the combination comprises:
4-{2-[5-methyl-1-(naphthalen-2-yl)-1H-pyrazol-3-yloxy]ethyl}morpholine hydrochloride and diclofenac or celecoxib; or
4-{2-[5-methyl-1-(naphthalen-2-yl)-1H-pyrazol-3-yloxy]ethyl}morpholine hydrochloride and metamizole; or
4-{2-[5-methyl-1-(naphthalen-2-yl)-1H-pyrazol-3-yloxy]ethyl}morpholine hydrochloride and ibuprofen or naproxen; or
4-{2-[5-methyl-1-(naphthalen-2-yl)-1H-pyrazol-3-yloxy]ethyl}morpholine hydrochloride and paracetamol.

29. A method of treatment and/or prophylaxis of a patient suffering from pain, or likely to suffer pain, the method comprising administering to the patient in need of such a treatment or prophylaxis a therapeutically effective amount of a combination as defined in claim 17 comprising at least one Sigma ligand and at least one Non-steroidal Anti-inflammatory Drug (NSAID).

30. The method according to claim 29 wherein the pain is selected from peripheral neuropathic pain, allodynia, causalgia, hyperalgesia, hyperesthesia, hyperpathia, neuralgia, neuritis and neuropathy.

31. The method according to claim 29 wherein the pain is post-operative pain.

32. A method for potentiating the analgesic effect of an NSAID when said NSAID is administered in the prophylaxis and/or treatment of pain, the method comprising administering to a patient in need thereof a therapeutically effective amount of a Sigma ligand as defined in claim 17 or a pharmaceutically acceptable salt, isomer, prodrug or solvate thereof.
